(19) United States
(12) Patent Application Publication
(10) Pub. No.: US 2023/0020563 A1
(43) Pub. Date: Jan. 19, 2023

(54) FLUID REMOVAL DEVICE

(71) Applicant: CM Technologies, Inc., San Diego, CA (US)

(72) Inventors: Amit Kumar SHARMA, New Delhi (IN); Nishith CHASMAWALA, Surat (IN); Geetika GARG, Ghaziabad (IN); Shreyas DIGHE, Nagpur (IN)

(21) Appl. No.: 17/651,622

(22) Filed: Feb. 18, 2022

Related U.S. Application Data
(60) Provisional application No. 63/222,668, filed on Jul. 16, 2021.

(51) Int. Cl. A61F 5/443; A61F 5/44; A61F 5/453; A61M 1/00

(52) U.S. Cl. CPC A61F 5/443; A61F 5/4405; A61F 5/453; A61M 1/82

(57) ABSTRACT

The invention comprises a urine removal devices that enable urine discharged from a male patient's penis to be contained within a flexible pouch and drawn out of the pouch and away from the genital region of the patient through a drain tube. The pouch comprises a first fluid compartment, a second fluid compartment and a fluid transmission passageway connecting the first fluid compartment and the second fluid compartment—wherein the fluid transmission passageway comprises a thin-film unidirectional fluid flow valve configured to enable for fluid flow from the first fluid compartment to the second fluid compartment, and to prevent fluid flow from the second fluid compartment to the first fluid compartment. The pouch may have an aperture sized to permit a male patient's penis to enter the pouch within the first fluid compartment.

### CROSS REFERENCE TO RELATED APPLICATIONS

This application claims the benefit of U.S. Provisional Patent Application No. 63/222,668, filed Jul. 16, 2021 (DAS Access Code: 4784), the entirety of which is incorporated by reference herein for all purposes.

### INCORPORATION BY REFERENCE

All publications and patent applications mentioned in this specification are incorporated herein by reference in their entirety to the same extent as if each individual publication or patent application was specifically and individually indicated to be incorporated by reference.

### TECHNICAL FIELD

The present disclosure generally relates to devices and systems of hygiene products that enable urine collection and disposal and, more particularly, to non-vacuum assisted devices and systems that enable fluid to be conveniently and hygienically removed from the region surrounding a patient's external genitalia area.

### BACKGROUND

The present disclosure relates to the management of urine output. Urine management systems are some of the most commonly used products in a variety of healthcare settings. Though typically associated with urinary incontinence in bedridden patients, the need extends further. For example, patients may require monitoring of urine output for clinicians to evaluate their fluid-levels. Urine management systems may be used to reduce the burden of frequent urination in those who are semi-ambulatory, or they may be used to reduce the risk of wound development by keeping the perineal and sacral skin dry. As the range of clinical needs is broad, all care settings, from an ICU to the home, may incorporate an assortment of urine management products.

One of the most common devices used for urine management in such patients is the indwelling urinary catheter, which may be designed for intermittent or extended use. These devices are low profile latex or silicon tubes inserted through the urethra, all the way into the bladder, where they are anchored using a balloon, to continually drain urine into a collection bag or container. Since the invention of the Foley catheter nearly 100 years ago, the extended use of indwelling catheters has continuously risen, until recently.

Placement of any product into the human body involves risks, especially when the target anatomy is sensitive or sterile. In the case of urinary catheters, the risk of patient harm during insertion and use is significant enough to require trained care providers such as nurses or physicians to perform the insertion, removal, and management of the devices. Even when caution is taken, indwelling catheters can cause significant impairment to urethral tissues during placement. Furthermore, maintaining catheter sterility is difficult. Patients often experience pain and bleeding during insertion and are faced with the possibility of bacteria being introduced into the bladder and renal system. Ultimately, there is a risk of patients developing injuries or urinary tract infections leading to subsequently bladder, kidney, or bloodstream infections.

To reduce these risks and improve clinical outcomes, care providers are transitioning away from indwelling catheters and increasingly using external management systems. These external management systems, especially in men, typically include a collection member to receive urine and an anchoring mechanism, sometimes in the form of an adhesive for securing the collection member in-place. Some of these systems are designed using soft and flexible materials for the collection member and include a drainage tube to remove accumulated fluid. Some external urine management devices used in male patients anchor either on the tip of the penile shaft or along the length of the shaft. In addition to a lack of accommodating anatomical size variations, itchiness, foreign body sensation, or feeling of wetness are some of these systems' shortcomings.

Using soft and flexible materials for the collection member, as well as other components of such a system, such as a drainage tube that may be used in a genital or perineal region, is important for multiple reasons. One reason is that skin and tissues in the genital and perineal regions are more sensitive than other areas of the body, meaning they are often at increased risk of injury. In clinical settings, it is not uncommon for patients to experience skin maceration, dermatitis, and pressure injuries. These complications may be due to tissue swelling exacerbating skin weakness, from improper movement along with extended exposure to moisture, or the use of devices/products in the area that become lodged between the skin and another surface. Furthermore, soft and flexible materials allow patients to move with less discomfort, and more flexible parts allow movement of a part of the product without the adhesive (used to secure the collection member) becoming pulled on or stressed.

Urine collection systems often irritate the skin and cause discomfort. When a portion of a management device is in contact with the human body, for example, in instances where the adhesive section is coupled to a genital area, the skin exposed to the internal aspect of the enclosed space of the collection section will become moist. Urine received by the collection section will either contact the skin directly or wet at least part of the internal surface of the flexible wall. Though the urine will be drained from the collection section periodically or continually, small amounts of moisture will continue to cling to the surfaces that were exposed to urine. Furthermore, skin (e.g., on the penis) constantly exudes small amounts of moisture via sweat, which over time, will accumulate inside an enclosed area. Either of these mechanisms of moisture introduction can leave liquid droplets in direct contact with the skin and increase the humidity within the enclosed space while potentially weakening the adhesive seal to the skin. This liquid/humidity creates a moist environment known to cause skin maceration, which reduces the structural integrity of the skin and increases the risk for skin damage such as dermatitis and pressure or friction injuries.

Initial stages of maceration are typically identified via a whitened appearance, wrinkling, and minor swelling. It is also known that changes in stratum corneum hydration, dermis hydration, transepidermal water loss (TEWL), skin pH, and skin hardness can be used to evaluate skin integrity. Healthy skin and macerated skin have measurable differences in these physiological markers, as well as more nuanced markers such as erythema index (EI) and white index (WI). Removal of moisture from the collection section and consequently from the skin reduces the risk that these markers deviate from healthy levels.

Currently available collection/protection devices that are external to the body rely on an absorbent material, which is a relic of diapers and pads. The use of the absorbent materials may lead to wetness of the skin, bulkiness, and dislodgement or peeling-off of the device. Retaining absorbed urine may also lead to inefficient suction and reduced air flow. Furthermore, the multitude of components and complex material layups can increase manufacturing complexity and cost.

As such, there is a need for a low-cost urine removal device that enables urine to be conveniently and hygienically removed from the region surrounding a male patient's external genitalia and that provides a dry internal compartment with little or no pooled or residual urine.

### SUMMARY

The invention provides a urine removal device that enables urine to be conveniently and hygienically removed from the region surrounding a male patient's external genitalia and that eliminates or substantially reduces pooling or collection of urine in the vicinity of the male patient's genitalia.

In an embodiment, the invention comprises a urine removal device comprising a flexible pouch having a proximal end and a distal end, an aperture disposed on an external surface of the pouch, wherein the aperture is capable of receiving at least a part of a penis, and an outlet conduit member affixed to the flexible pouch, the outlet conduit member comprising a fluid inlet, a fluid outlet, and a lumen connecting the fluid inlet to the fluid outlet. The flexible pouch comprises a fluid tight first fluid compartment, a fluid tight second fluid compartment and a first fluid transmission passageway interconnecting the first fluid compartment and the second fluid compartment. The aperture disposed on the external surface of the pouch is an aperture into the first fluid compartment. The first fluid transmission passageway comprises a first thin-film unidirectional fluid flow valve that permits fluid flow from the first fluid compartment to the second fluid compartment and that interferes with fluid reflux from the second fluid compartment to the first fluid compartment. The first fluid compartment is positioned closer to the proximal end of the pouch and further from the distal end of the pouch. The second fluid compartment is positioned closer to the distal end of the pouch and further from the proximal end of the pouch.

The thin-film unidirectional fluid flow valve may comprise a flutter valve.

In an embodiment of the urine removal device, the fluid inlet of the outlet conduit member is located within the second fluid compartment and the fluid outlet of the outlet conduit member is configured for coupling with a fluid collection receptacle.

In another embodiment of the urine removal device, the first fluid transmission passageway is sized to enable fluid flow from the first fluid compartment to the second fluid compartment at a first flow rate, the outlet conduit member is sized to enable fluid flow out from the second fluid compartment at a second flow rate, and the first flow rate is greater than the second flow rate.

The first fluid transmission passageway may be sized to enable fluid flow from the first fluid compartment to the second fluid compartment at a flow rate of 10 ml per min or more.

In a particular embodiment, the first fluid transmission passageway comprises an fluid passageway inlet and a fluid passageway outlet, each positioned between the aperture and the fluid inlet of the outlet conduit member.

The external peripheral boundaries of the first fluid compartment may be located partially or wholly within the external peripheral boundaries of the second fluid compartment.

The urine removal device may include an air exchanger that is gas permeable and liquid impermeable, and that is configured to permit air exchange from outside the pouch and into the first fluid compartment, and from the first fluid compartment to outside the pouch.

The urine removal device may include a fold-resistant feature on the outlet conduit member, wherein the fold-resistant feature is configured to resist the collapse of a lumen defined by the outlet conduit member.

In one embodiment of the urine removal device, the fold-resistant feature comprises one or more accordion pleats or bellows folds.

In a specific embodiment of the urine removal device, a plurality of flow directors are disposed between at least two internal surfaces of the pouch or the first fluid compartment or the second fluid compartment, such that when the two internal surfaces are adjacent each other, channels are created therebetween.

In another embodiment of the urine removal device, the fluid inlet of the outlet conduit member may be positioned between a fluid outlet of the first fluid transmission passageway and the distal end of the pouch. The outlet conduit member may be coupled to the fluid collection receptacle by a drain tube, wherein the drain tube has one or more capillary structures therewithin.

An open-foam cell assembly may be positioned as a fluid transmission intermediate between the outlet conduit member and the drain tube, wherein the open-cell foam assembly defines a plurality of fluid channels, each of said fluid channels interconnecting the outlet conduit member and the drain tube.

In an embodiment, a suction bulb may be positioned as a fluid transmission intermediate between the outlet conduit member and the drain tube, and wherein the suction bulb has a fluid inlet coupled with the outlet conduit member, and has a fluid outlet coupled with an inlet of the fluid collection receptacle.

The urine removal device may comprise a fluid tight third fluid compartment wherein the third fluid compartment is positioned as an intermediate fluid compartment between the second fluid compartment and the outlet conduit member. A second fluid transmission passageway interconnects the second fluid compartment and the third fluid compartment, the second fluid transmission passageway comprising a second thin-film unidirectional fluid flow valve that permits fluid flow from the second fluid compartment to the third fluid compartment and that interferes with fluid reflux from the third fluid compartment to the second fluid compartment. The fluid inlet of the outlet conduit member may be located within the third fluid compartment and the fluid outlet of the outlet conduit member is configured for coupling with a fluid collection receptacle.

In an embodiment of the urine removal device, the second thin-film unidirectional fluid flow valve is a flutter valve.

The urine removal device may additionally comprise an adhesive patch attached to the flexible pouch, the adhesive patch configured for fastening the flexible pouch to a patient's suprapubic region.

In an alternate embodiment, the invention provides a urine removal device comprising a flexible pouch having a proximal end and a distal end, an aperture disposed on an external surface of the pouch, wherein the aperture is capable of receiving at least a part of a penis, and an outlet conduit member affixed to the flexible pouch, the outlet conduit member comprising a fluid inlet, a fluid outlet, and a lumen connecting the fluid inlet to the fluid outlet. The flexible pouch comprises a fluid tight first fluid compartment, a fluid tight second fluid compartment, a first fluid transmission passageway interconnecting the first fluid compartment and the second fluid compartment, a fluid tight third fluid compartment positioned as an intermediate fluid compartment between the second fluid compartment and the outlet conduit member, and a second fluid transmission passageway interconnecting the second fluid compartment and the third fluid compartment. The aperture disposed on the external surface of the pouch is an aperture into the first fluid compartment. The first fluid transmission passageway comprises a first thin-film unidirectional fluid flow valve that permits fluid flow from the first fluid compartment to the second fluid compartment and that interferes with fluid reflux from the second fluid compartment to the first fluid compartment. The second fluid transmission passageway comprises a second thin-film unidirectional fluid flow valve that permits fluid flow from the second fluid compartment to the third fluid compartment and that interferes with fluid reflux from the third fluid compartment to the second fluid compartment. The first fluid compartment is positioned closer to the proximal end of the pouch and further from the distal end of the pouch. The second fluid compartment is positioned closer to the distal end of the pouch and further from the proximal end of the pouch. The fluid inlet of the outlet conduit member is located within the third fluid compartment and the fluid outlet of the outlet conduit member is configured for coupling with a fluid collection receptacle.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A illustrates a plan view of an embodiment of a urine removal device in accordance with the present invention.

FIG. 1B illustrates a plan view of another embodiment of a urine removal device in accordance with the present invention.

FIGS. 1C and 1D, illustrate a perspective view and an exploded view of an example embodiment of a urine removal device.

FIGS. 1E and 1F, illustrate a perspective view and an exploded view of another example embodiment of a urine removal device.

FIG. 2A illustrates the flow pattern through a urine removal device.

FIGS. 2B-2C illustrate a prone patient wearing a urine removal device according to an example embodiment.

FIG. 3 illustrates a urine management system according to an example embodiment.

FIGS. 4A, 4B, 4C and 5A illustrate perspective views of a urine removal device having flow directors on internal walls according to an example embodiment.

FIGS. 5B, 5C, and 5D illustrate fibers oriented on a surface at angles of 0 deg., 15 deg., and 90 deg, respectively.

FIG. 5E illustrates a wall having a flow director layer.

FIG. 5F illustrates a wall having a flow director layer and an opposing wall in contact with the flow director layer.

FIG. 6 illustrates air and fluid flow patterns through a urine removal device.

FIGS. 7A to 7D illustrate embodiments of air exchanger(s) provided on or within a urine removal device.

FIGS. 8 and 9 illustrate embodiments of a fluid transmission passageway formed within a urine removal device.

FIG. 10 illustrates capillary structures for drawings fluid out of a urine removal device.

FIGS. 11A to 11F illustrate a bell siphon assembly within a urine removal device.

FIG. 12 illustrates a urine removal device having a foam assembly provided to prevent occlusion of outlet conduit(s).

FIGS. 13A and 13B illustrate a suction bulb assembly to expel fluid out of a urine removal device and into a urine collection receptacle.

FIGS. 13A and 13B illustrate a suction bulb assembly to expel fluid out of a urine removal device and into a urine collection receptacle.

FIGS. 13C and 13D illustrate an alternative embodiment of a urine removal device having an assembly for expelling fluid and for eliminating urine reflux into a fluid compartment in which a patient's penis is positioned.

FIG. 13E illustrates another embodiment of a urine removal device having an assembly for expelling fluid and for eliminating urine reflux into a fluid compartment in which a patient's penis is positioned.

FIGS. 14A to 14C illustrate an assembly for preventing kinking or occlusion of outlet(s) in a urine removal device.

FIG. 15A illustrates a perspective view of an adhesive patch according to an example embodiment.

FIG. 15B illustrates a perspective view of the adhesive patch of FIG. 15A showing various layers according to an example embodiment.

FIG. 16 illustrates a perspective view of an adhesive patch having perforations according to another example embodiment.

FIG. 17 illustrates a perspective view of an adhesive patch having perforations showing various layers according to another example embodiment.

FIG. 18 illustrates a perspective view of an adhesive patch having perforations showing various layers according to another example embodiment.

FIGS. 19A-19L illustrates various adhesive patch shapes according to example embodiments.

FIG. 20 illustrates an adhesive patch shape according to another example embodiment.

FIG. 21 illustrates a plan view of the adhesive patch of FIG. 15A.

FIGS. 22 to 24 illustrate adhesive patch shapes according to other example embodiments.

FIG. 25 illustrates a device having a removable attachment for the pouch.

FIG. 26 illustrates a device having a removable attachment to a clothing.

### DETAILED DESCRIPTION OF THE INVENTION

The embodiments disclosed herein provide urine removal devices that enable urine to be conveniently and hygienically removed from the region surrounding a male patient's external genitalia area. The urine removal devices may be suction assisted or suction unassisted.

For the purposes of this disclosure, the term “proximal” and “distal” are used with reference to the length of the urine removal device or the length of a pouch that forms part of the urine removal device; that is, “proximal” denotes the first end of the device or pouch toward the suprapubic region beyond where it engages with (or is positioned in the vicinity of) the base of the penis and “distal” denotes the opposed second end of the device or pouch toward the end where it resides between the legs of the patient. For the purposes of this disclosure, the terms “top” and “bottom” are used with reference to upper and lower surfaces of the urine removal device or components thereof. That is, “top” denotes a surface or direction that is coincident with or in the direction of the upper surface of the penis. In contrast, “bottom” denotes a surface or direction that is coincident with or in the direction of the underside of the penis.

This disclosure describes urine removal devices that enable urine discharged from a male patient's penis to be contained within a flexible pouch and drawn out of the pouch and away from the genital region of the patient through a drain tube—which may optionally be connected to a source of suction pressure or negative pressure. The pouch comprises a first fluid compartment, a second fluid compartment and a fluid transmission passageway connecting the first fluid compartment and the second fluid compartment—wherein the fluid transmission passageway comprises a thin-film unidirectional fluid flow valve configured to enable for fluid flow from the first fluid compartment to the second fluid compartment, and to prevent fluid flow from the second fluid compartment to the first fluid compartment. In an embodiment, the thin-film unidirectional fluid flow valve is a flutter valve. The fluid transmission passageway comprises a fluid inlet, a fluid outlet and a lumen that is formed from one or more collapsible flexible sheets. The fluid inlet of the fluid transmission passageway is provided towards or at a distal end of the first fluid compartment. The fluid outlet of the fluid transmission passageway is provided within the second fluid compartment. The lumen connects the fluid inlet and the fluid outlet and forms a fluid passageway therebetween. In an embodiment, the fluid transmission passageway is sized to enable fluid flow from the first fluid compartment to the second fluid compartment at a flow rate of at least 10 ml per min. The first fluid compartment is positioned relatively closer to the proximal end of the pouch and relatively further from the distal end of the pouch, whereas the second fluid compartment is positioned relatively closer to the distal end of the pouch and relatively further from the proximal end of the pouch. In various embodiments the pouch may have an aperture sized to permit a male patient's penis to enter the pouch within the first fluid compartment.

The pouch and each of the fluid compartments may be formed from a plurality of collapsible flexible sheets that are affixed together or sealed together along the peripheries of the pouch and/or first or second fluid compartments. The flexible sheets may comprise one or more fluid-tight (liquid-impermeable) materials (for example, polyvinyl chloride, polypropylene, polyethylene, low-density polyethylene, high-density polyethylene, ethylene-vinyl acetate, polyvinylidene dichloride, biaxially oriented polypropylene, ethylene vinyl alcohol, natural rubber latex, silicon rubber, polyurethane, coated synthetic or natural fabric, etc.) wherein the fluid-tight materials define internal compartments for the pouch and the first and second fluid compartments therewithin. The fluid-tight materials form one or more external surfaces and internal surfaces for the pouch and the fluid compartments. The aperture may be fixedly or adaptably sized to enable a male patient's penis to be inserted therethrough and to be positioned within the first fluid compartment. Additionally, each of the pouch, first fluid compartment and aperture are configured such that upon insertion of the patient's penis, at least a part of the penis (for example, at least the tip of the penis or the meatus) and optionally, substantially the whole of the penis, is housed within the first fluid compartment in the region between the aperture and the second fluid compartment, and/or between the aperture and the distal end of the pouch.

The urine removal device may include one or more fasteners or anchors that enable the penis to remain housed within the pouch unless intentionally removed (or removed by application of force).

In addition, the urine removal device also includes one or more outlet conduit members. The one or more outlet conduit members are positioned in the second fluid compartment at a distal end of the second fluid compartment, such that said conduit member(s) enable urine that is delivered into the second fluid compartment from the first fluid compartment (through the fluid transmission passage) to flow out from, or be drawn out of, the second fluid compartment. Each of the one or more outlet conduit members comprises a conduit shaft having one or more fluid inlets, one or more fluid outlets, and a lumen connecting each of the inlet(s) and outlet(s). The one or more fluid inlets of the outlet conduit member(s) are positioned within or in fluid communication with the second fluid compartment of the pouch. The one or more fluid outlets of the outlet conduit member(s) are configured for direct or indirect coupling with a urine collection bag—for example by means of a drain tube. The one or more fluid inlets, one or more fluid outlets and lumen(s) connecting the two, together define one or more fluid passageways between the internal compartment and the one or more fluid outlet(s) (or a receptacle to which the one or more fluid outlet(s) are connected).

In some embodiments, the one or more such conduit members are configured such that one or more fluid inlet (s) are positioned within or in fluid communication with the second fluid compartment of the pouch, between a fluid outlet of the fluid transmission passageway and the distal end of the pouch. Further, the fluid transmission passageway between the first fluid compartment and the second fluid compartment is formed such that each of the fluid inlet and the fluid outlet of said fluid transmission passageway are positioned between the aperture and the one or more fluid inlets of the one or more conduit members. In a particular embodiment, the fluid transmission passageway is sized to enable fluid flow from the first fluid compartment to the second fluid compartment at a first flow rate, and the outlet conduit member(s) in the second fluid compartment is/are sized so as to enable fluid flow out from the second fluid compartment at a second flow rate, wherein the first flow rate is greater than the second flow rate.

In one or more embodiments, one or more of the conduit members are provided with an occlusion resistant feature or an anti-kinking feature, comprising one or more accordion pleats or bellows folds at one or more regions of the conduit members. The accordion pleats or bellows folds enable the conduit members to resist being kinked, twisted, folded, or collapsed, which maintains the patency of the lumen defined between the fluid inlet(s) and fluid outlet(s) of the conduit member(s) and prevents blockage of the fluid passageway between the fluid inlet(s) and fluid outlet(s). In an embodiment, the occlusion resistant feature or anti-kinking feature may be on the one or more on the conduit shaft(s) of the conduit members at a location outside of the pouch (or outside of the second fluid compartment) and may be located between the distal end of the pouch and a fluid outlet of the conduit member.

The pouch is additionally provided with one or more air exchangers (other than the aperture) formed so as to enable air to be transmitted through the air exchanger(s) into and out of the first fluid compartment (i.e. that is gas permeable) but which simultaneously resist transmission of liquid through the exchanger(s) (i.e. that is liquid impermeable), thereby preventing urine within the first fluid compartment from being expelled via the air exchanger. In an embodiment, the one or more air exchangers are positioned relatively closer to the proximal end of the first fluid compartment and relatively further from the distal end of the first fluid compartment. The one or more air exchangers enable air circulation in the first fluid compartment—thereby enabling the first fluid compartment to dry out quickly even after urine has been discharged into the first fluid compartment by a subject wearing the urine removal device. When the urine removal device is disposed on a subject's penis (i.e., when at least some of the penis is housed within the first fluid compartment of the pouch), urine released from the meatus flows into the first fluid compartment and is expelled by the action of fluid pressure from the first fluid compartment through the fluid transmission passageway into the second fluid compartment.

Since the fluid transmission passageway comprises a thin-film unidirectional fluid flow valve or a flutter valve, urine reflux from the second fluid compartment back into the first fluid compartment is prevented—despite any movement or change in orientation of the urine removal device, and urine that is delivered to the second fluid compartment from the first fluid compartment flows out (for example, by action of fluid pressure or gravity) or is drawn out (for example by capillary action or suction) through the one or more outlet conduit members into a urine collection receptacle that is external to the pouch (for example into a receptacle to which the one or more outlet conduit members are connected). As a result, urine released or pooled into the pouch is readily and hygienically removed from the pouch and the vicinity of the patient's penis. In certain embodiments where suction is capable of being applied to the one or more outlet conduit members, as a result of the application of suction through the outlet conduit member(s), negative pressure is created within the second fluid compartment, and consequently in the first fluid compartment, and air is drawn into the pouch through the one or more air exchangers for as long as suction is applied. The air flow thus generated additionally serves to draw urine from the first fluid compartment, into the second fluid compartment, and thereafter, out through the one or more outlet conduit members.

Specific embodiments of the invention are now described in connection with the accompanying figures in which like reference characters refer to the same parts throughout the different views. The drawings are not necessarily to scale, emphasis instead being placed upon illustrating embodiments of the present invention.

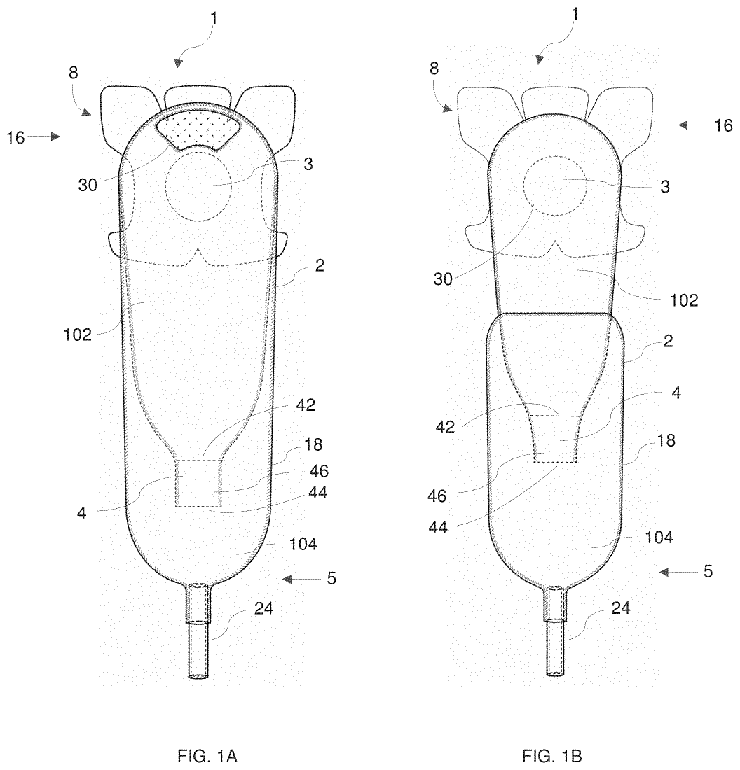

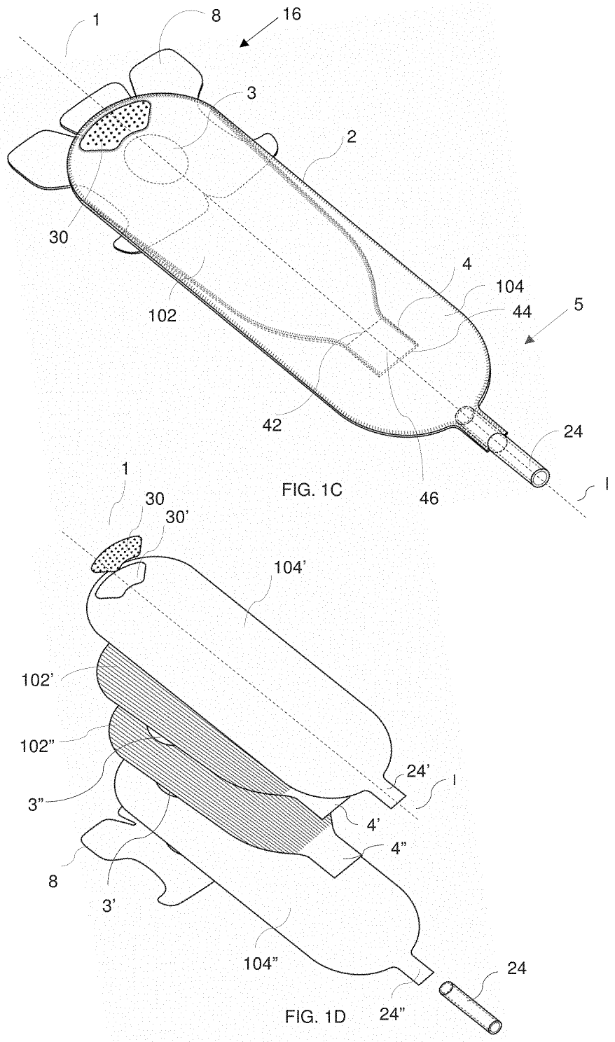

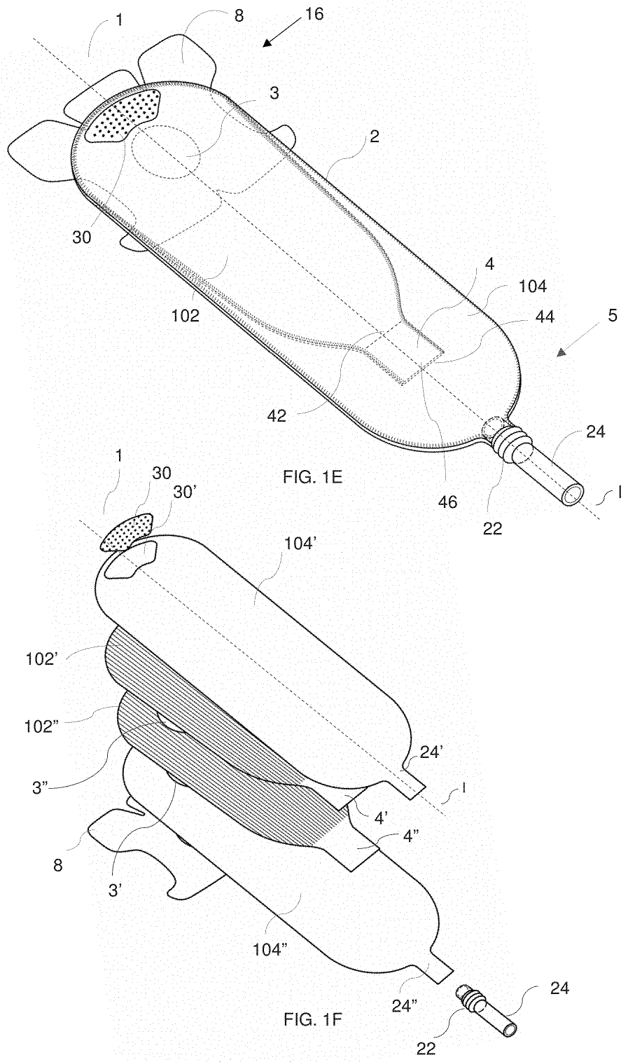

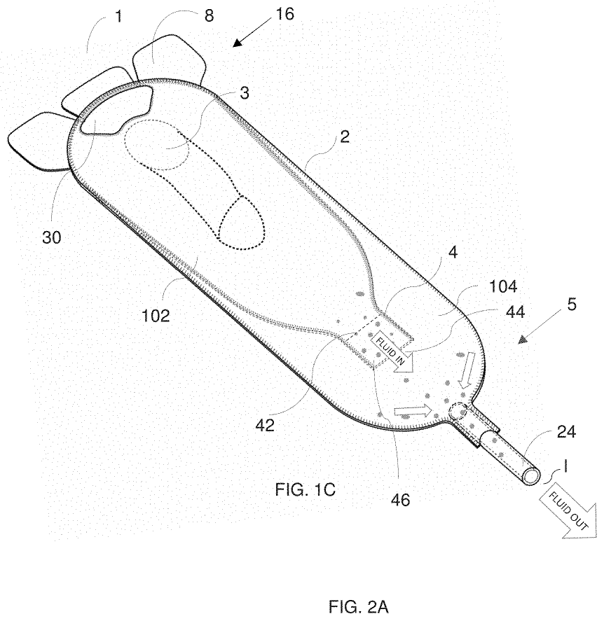

Referring to FIG. 1A-1F, a urine removal device 1 is shown according to example embodiments. FIGS. 1A and 1B show top plan views of two different embodiments of a urine removal device 1. In a first embodiment illustrated in FIG. 1A, urine removal device 1 comprises a pouch 2 having a first fluid compartment 102 and a second fluid compartment 104, wherein the external peripheral boundaries of first fluid compartment 102 lies substantially within the external peripheral boundaries of second fluid compartment 104. In a second embodiment illustrated in FIG. 1B, urine removal device 1 comprises a pouch 2 having a first fluid compartment 102 and a second fluid compartment 104, wherein the external peripheral boundaries of first fluid compartment 102 lie partially within the external peripheral boundaries of second fluid compartment 104 and partially outside the external peripheral boundaries of second fluid compartment 104.

FIG. 1C is a perspective view of the urine removal device 1 as previously illustrated in FIG. 1A, and FIG. 1D shows another perspective view of the urine removal device 1 (of FIG. 1C) exploded to show the interaction of the components more clearly. FIG. 1E provides a perspective view of an alternate embodiment of urine removal device 1, and FIG. 1F shows an exploded view of this alternate embodiment of FIG. 1E.

The urine removal device 1, as shown in the embodiments illustrated in FIGS. 1A to 1F, includes a pouch 2 having a first fluid compartment 102 and a second fluid compartment 104. Each fluid compartment 102, 104 may comprise a fluid compartment defined or formed by a plurality of flexible sheets joined together at one or more peripheral boundaries of said fluid compartment. It would be understood that in other embodiments, each fluid compartment 102, 104 may be formed of a single continuous side wall having two opposite surfaces, where one of the two opposite surfaces forms an external sidewall and the other of the two opposite surfaces forms an internal sidewall. The pouch 2 has a proximal end 16 and a distal end 5.

The pouch 2 additionally comprises a fluid transmission passageway 4 connecting the first fluid compartment 102 and the second fluid compartment 104. In an embodiment, the fluid transmission passageway 4 comprises a thin-film unidirectional fluid flow valve or a flutter valve configured to enable fluid flow from the first fluid compartment 102 to the second fluid compartment 104, and to prevent fluid flow from the second fluid compartment 104 to the first fluid compartment 102. The fluid transmission passageway 4 comprises a fluid inlet 42, a fluid outlet 44 and a lumen 46 that is formed from one or more collapsible flexible sheets. The fluid inlet 42 of the fluid transmission passageway 4 is provided towards or at a distal end of the first fluid compartment 102. The fluid outlet 44 of the fluid transmission passageway 4 is provided within the second fluid compartment 104. The lumen 46 connects the fluid inlet 42 and the fluid outlet 44 and forms a fluid passageway therebetween. In an embodiment, the fluid transmission passageway 4 is sized to enable fluid flow from the first fluid compartment 102 to the second fluid compartment 104 at a flow rate of at least 10 ml per min.

The first fluid compartment 102 is positioned relatively closer to the proximal end 16 of the pouch 2 and relatively further from the distal end 5 of the pouch 2, whereas the second fluid compartment 104 is positioned relatively closer to the distal end 5 of the pouch 2 and relatively further from the proximal end 16 of the pouch 2. In various embodiments the pouch 2 may have an aperture 3 sized to permit a male patient's penis to enter the pouch 2 within the first fluid compartment 102. The pouch 2 and each of the fluid compartments 102, 104 may be formed from a plurality of collapsible flexible sheets that are affixed together or sealed together along the peripheries of the pouch 2 and/or first or second fluid compartments 102, 104. The flexible sheets may comprise one or more fluid-tight (liquid-impermeable) materials (for example, polyvinyl chloride, polypropylene, polyethylene, low-density polyethylene, high-density polyethylene, ethylene-vinyl acetate, polyvinylidene dichloride, biaxially oriented polypropylene, ethylene vinyl alcohol, natural rubber latex, silicon rubber, polyurethane, coated synthetic or natural fabric, etc.) wherein the fluid-tight materials define internal compartments for the pouch 2 and the first and second fluid compartments 102, 104 therewithin. The fluid-tight materials form one or more external surfaces and internal surfaces for the pouch.

The aperture 3 may be fixedly or adaptably sized to enable a male patient's penis to be inserted therethrough and to be positioned within the first fluid compartment 102. Additionally, each of the pouch 2, first fluid compartment 102 and aperture 3 are configured such that upon insertion of the patient's penis, at least a part of the penis (for example, at least the tip of the penis or the meatus) and optionally, substantially the whole of the penis, is housed within the first fluid compartment 102 in the region between the aperture 3 and the second fluid compartment 104, and/or between the aperture 3 and the distal end 5 of the pouch 2.

The urine removal device 1 may include one or more fasteners or anchors (for example an adhesive patch 8) that enable the penis to remain housed within the pouch 2 unless intentionally removed (or removed by application of force). The fastener or anchor may be attached to an external surface of the pouch 2 to secure the pouch 2 to the patient's penis, abdomen, scrotum, or suprapubic region. In an embodiment where the fastener or anchor is an adhesive patch 8, the adhesive patch may be a single layer, or it may be comprise of several layers.

In addition, the urine removal device 1 also includes one or more outlet conduit members 24. The one or more outlet conduit members 24 are positioned in the second fluid compartment 104 at a distal end 5 of the second fluid compartment 104, such that said conduit member(s) 24 enable urine that is delivered into the second fluid compartment 104 from the first fluid compartment 102 (through the fluid transmission passageway 4) to flow out from, or be drawn out of, the second fluid compartment 104. Each of the one or more outlet conduit members 24 comprises a conduit shaft having one or more fluid inlets, one or more fluid outlets, and a lumen connecting each of the inlet(s) and outlet(s). The one or more fluid inlets of the outlet conduit member(s) 24 are positioned within or in fluid communication with the second fluid compartment of the pouch. The one or more fluid outlets of the outlet conduit member(s) 24 are configured for direct or indirect coupling with a urine collection bag (for example, by means of an interconnecting drain tube). The one or more fluid inlets, one or more fluid outlets and lumen(s) connecting the two, together define one or more fluid passageways between the second fluid compartment 104 and the one or more fluid outlet(s) (or a receptacle to which the one or more fluid outlet(s) are connected).

In some embodiments, the one or more such outlet conduit member(s) 24 are configured such that one or more fluid inlet (s) are positioned within or in fluid communication with the second fluid compartment 104 of the pouch 2, between a fluid outlet 44 of the fluid transmission passageway 4 and the distal end 5 of the pouch 2. Further, the fluid transmission passageway 4 between the first fluid compartment 102 and the second fluid compartment 104 is formed such that each of the fluid inlet 42 and the fluid outlet 44 of said fluid transmission passageway 4 are positioned between the aperture 3 and the one or more fluid inlets of the one or more outlet conduit member(s) 24. In a particular embodiment, the fluid transmission passageway 4 is sized to enable fluid flow from the first fluid compartment 102 to the second fluid compartment 104 at a first flow rate, and the outlet conduit member(s) 24 in the second fluid compartment 104 is/are sized so as to enable fluid flow out from the second fluid compartment 104 at a second flow rate, wherein the first flow rate is greater than the second flow rate.

In an embodiment, a conduit shaft of the one or more outlet conduit member(s) 24 may comprise a rigid, partially rigid, or partially flexible conduit shaft and may be positioned within or affixed to the pouch 2 and/or the second fluid compartment 104.

In one or more embodiments (for example, the embodiments shown in FIGS. 1E and 1F), one or more of the outlet conduit member(s) 24 are provided with an occlusion resistant feature or an anti-kinking feature, comprising one or more accordion pleats or bellows folds 22 at one or more regions of the outlet conduit member(s) 24. The accordion pleats or bellows folds 22 enable the outlet conduit member(s) 24 to resist being kinked, twisted, folded, or collapsed, which maintains the patency of the lumen defined between the fluid inlet(s) and fluid outlet(s) of the outlet conduit member(s) 24 and prevents blockage of the fluid passageway between the fluid inlet(s) and fluid outlet(s). In an embodiment, the occlusion resistant feature or anti-kinking feature may be on the one or more on the conduit shaft(s) of the outlet conduit member(s) 24 at a location outside of the pouch 2 and may be located between the distal end of the pouch 2 and a fluid outlet of the outlet conduit member(s) 24.

The pouch 2 is additionally provided with one or more air exchangers 30 (other than aperture 3) formed to enable air to be transmitted through the air exchanger(s) 30 into and out of the first fluid compartment 102 but which simultaneously resist transmission of liquid through the air exchanger(s) 30, thereby preventing urine within the first fluid compartment 102 from being expelled via the air exchanger(s) 30. In an embodiment, the one or more air exchanger(s) 30 are positioned relatively closer to the proximal end of the first fluid compartment 102 and relatively further from the distal end of the first fluid compartment 102. The one or more air exchanger(s) 30 enable air circulation in the first fluid compartment 102—thereby enabling the first fluid compartment 102 to dry out quickly even after urine has been discharged into the first fluid compartment 102 by a subject wearing the urine removal device 1. In an embodiment, the air exchanger(s) 30 may comprise a patch or window formed of an air permeable, and liquid impermeable surface or fabric or substrate, that is formed on a surface of the first fluid compartment 102—such that one surface of the air exchanger 30 forms a portion of an internal surface of the first fluid compartment 102, and a second surface of the air exchanger 30 forms a portion of an external surface of the pouch 2.

When the urine removal device 1 is disposed on a subject's penis (i.e., when at least some of the penis is housed within the first fluid compartment 102 of the pouch 2), urine released from the meatus flows into the first fluid compartment 102 and is expelled by the action of fluid pressure from the first fluid compartment 102 through the fluid transmission passageway 4 into the second fluid compartment 104. Since the fluid transmission passageway 4 comprises a thin-film unidirectional fluid flow valve or a flutter valve, urine reflux from the second fluid compartment 104 back into the first fluid compartment 102 is prevented—despite any movement or change in orientation of the urine removal device 1, and urine that is delivered to the second fluid compartment 104 from the first fluid compartment 102 flows out (for example, by action of fluid pressure or gravity) or is drawn out (for example by capillary action or suction) through the one or more outlet conduit member(s) 24 into a urine collection receptacle that is external to the pouch 2 (for example into a receptacle to which the one or more outlet conduit member(s) 24 are connected).

As a result, urine released or pooled into the first fluid compartment 102 of pouch 2 is readily and hygienically removed from the pouch 2 and the vicinity of the patient's penis. In certain embodiments where suction is capable of being applied to the one or more outlet conduit member(s) 24, as a result of the application of suction through the outlet conduit member(s) 24, negative pressure is created within the second fluid compartment 104, and consequently in the first fluid compartment 102, and air is drawn into the pouch through the one or more air exchangers 30 for as long as suction/negative pressure is applied. The air flow thus generated additionally serves to draw urine from the first fluid compartment 102, into the second fluid compartment 104, and thereafter, out through the one or more outlet conduit member(s) 24.

The embodiment of the urine removal device 1 that is illustrated in FIG. 1A and FIG. 1C, is shown in an exploded view in FIG. 1D to further illustrate the relationship of the components and features. In the illustrated embodiment, the first fluid compartment 102 is formed by two flexible sheets 102′, 102″ joined together at peripheral boundaries, while second fluid compartment 104 is formed by two flexible sheets 104′, 104″ joined together at peripheral boundaries. The flexible sheets 102′, 102″ lie between the flexible sheets 104′, 104″ and as a result, the first fluid compartment is disposed within the peripheral boundaries of second fluid compartment 104. Each of flexible sheets 102″ and 104″ have an aperture 3″ and 3′ respectively formed therewithin and flexible sheets 102″ and 104″ are positioned relative to each other such that apertures 3″ and 3′ are substantially aligned with each other and thereby form an aperture through which a subject's penis can be inserted into first fluid compartment 102. The flexible sheets 102′, 102″ are each respectively shaped to include a flutter valve wall segment 4′, 4″, such that when joined together, they define a fluid transmission passageway 4 (i.e. a flutter valve) through which fluid within first fluid compartment 102 can pass into second fluid compartment 104.

The flexible sheets 104′, 104″ are each respectively shaped to include an outlet conduit member holder segment 24′, 24″, such that when joined together, they permit outlet conduit member 24 to be held securely between flexible sheets 104′, 104″. While the embodiments in FIGS. 1C and 1D illustrate the outlet conduit member 24 without an anti-kinking or anti-occlusion feature, FIGS. 1E and 1F illustrate an alternate embodiment where the outlet conduit member 24 includes an anti-kinking or occlusion resistant feature 22 of the kind described above.

Air exchanger 30 is disposed in an aperture 30′ formed in flexible sheet 104′. A corresponding aperture (not shown) is also formed in the flexible sheet 102′ that is adjacent to flexible sheet 104′, such that one surface of air exchanger 30 forms a forms a portion of an internal surface of the first fluid compartment 102, and a second surface of the air exchanger 30 forms a portion of an external surface of pouch 2 and/or the second fluid compartment 104.

In an embodiment of the urine removal device 1, a skin-friendly material disposed on one or more internal and/or external surfaces of pouch 2, for improved patient feel.

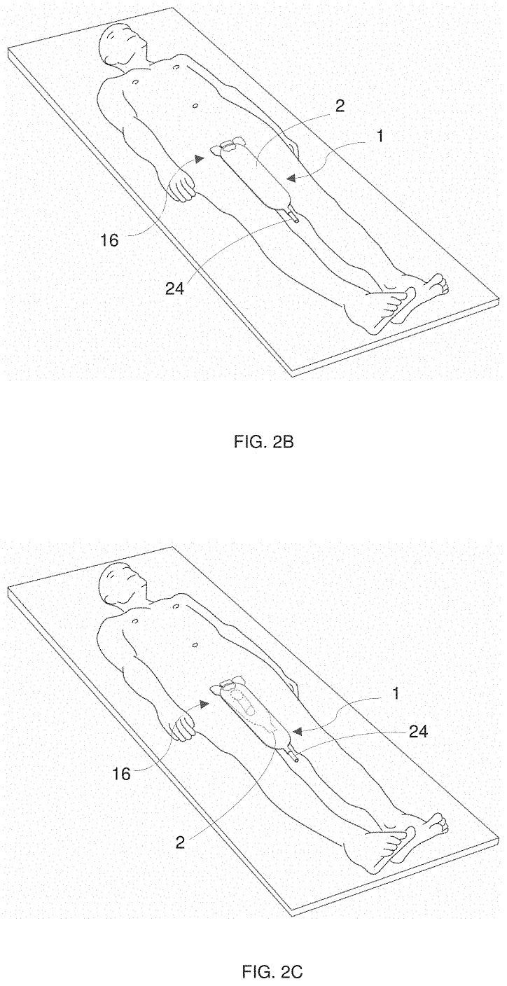

The urine removal device 1 is shown in exemplary patient use scenarios in FIGS. 2A-2C. In the above arrangements, the outlet conduit member 24, enables urine to flow out of second fluid compartment 104 from a distal end 5 of the pouch 2. For example, FIG. 2A illustrates the manner in which urine released into first fluid compartment 102 of pouch 2 of the urine removal device 1 (by a patient's penis when it is positioned within pouch 2 through the aperture 3) flows from the first fluid compartment 102 into the second fluid compartment 104 through fluid transmission passageway 4 and thereafter out of outlet conduit member 24.

In FIGS. 2B and 2C, the patient is shown laying supine with the urine removal device 1 attached. In this embodiment, the device 1 attaches around the penis such that the outlet conduit member 24 resides between the patient's legs.

FIG. 3 illustrates a further embodiment of the urine removal device 1, in an exemplary patient use scenario—wherein the outlet conduit member 24 connects to a urine collection bag 48 or receptacle by means of a drain tube 50. In the above arrangements, the outlet conduit member 24, enables urine to flow out of second fluid compartment 104 from a distal end 5 of the pouch 2 and into the urine collection bag 48.

Each fluid compartment 102, 104 of pouch 2 of the present invention is defined by a substantially flexible and liquid impermeable form with a fluid receiving area between one or more walls (for example a first wall and a second wall). The form creates an essentially enclosed space, either independently or when in communication with the body, and has a patient-side (second wall) and an environment-side (first wall). The form is configured to be liquid impermeable and essentially enclosed to provide a receiving area to receive urine and at least temporarily hold urine within the enclosed space. The impermeable form may be constructed of multiple layers, either coupled or uncoupled, and the entire construction of layers, whether single or multiple, may be described as a flexible wall.

The outermost aspect of each such flexible wall is composed of a liquid impermeable material to ensure any effluents received are temporarily contained within the form. The flexible wall is fashioned such that the internal aspect of the enclosed space will not allow the flexible form to collapse in a way that can fully or partially block fluid communication between the proximal and distal ends of the space. Fluid communication requires some viable flow channels between the surfaces and may be achieved with a variety of constructions. Furthermore, the flexible wall can be fabricated to reduce or eliminate the occurrence of a vacuum lock. In these instances, a low pressure pulls a surface against either the source itself in such a way to prevent fluid communication or pulls two or more surfaces against each other in a manner that creates a separate chamber surrounding the source resulting in a chamber that is no longer in fluid communication with the original enclosed space.

As described in more detail herein, example embodiments of the pouch 2 illustrate features for maintaining fluid communication between opposing ends of the liquid impermeable form such as by way of nonlimiting example: the addition of physical separation components running from one end of the form to another end or through a section of the form, the inclusion of material within the form involving physical characteristics such as webbing or mesh, texturing of the form internal or external surfaces, creating undulations or texture-like features by altering the form's cross-sectional shape, or utilizing a material and design that provides a shape whose structure is stronger than the forces that would collapse the form. One skilled in the art would recognize that there are many other ways to maintain fluid communication inside the pouch and such designs are within the spirit of the embodiments disclosed herein and contemplated in this disclosure.

“Flow directors,” as used herein refers to features on or within the pouch 2 that provide for fluid flow channels or pathways to provide for urine flow within and out of the pouch, for example when the urine removal device is exposed to gravity or applied with vacuum. The features may be at a small scale such as where capillary forces govern, or larger features, or combinations of small and large features, with for example, surface treatments that may affect hydrophobicity. The flow directors provide grooves (channels) when the pouch walls come together under vacuum, thus preventing vacuum lock between the layers of the pouch. In some embodiments the flow directors are oriented in the direction of the desired flow but in other embodiments flow directors may be at other angles or randomly arranged. The following figures and descriptions provide non-limiting examples of flow directors.

Flow directors can be formed from the same base material as that of the pouch layer or of different material which may be then bonded with the pouch layer using the process of heat or other method of lamination, gluing, chemical bonding, printing, or any other similar processes practiced in the art. When flow directors are made of same base material as that of the pouch layer, they can be formed during the primary extrusion of the pouch layer or later using a secondary process such as embossing, chemical etching, laser etching, grooving, engraving, carving, imprinting, etc. The flow directors may be left uncoated or coated with chemicals such as Teflon, PTFE, etc. or nanoparticles to improve hydrophobicity. The size of flow directors can range from few nanometers, as it would be in case of etching, to few microns as it would be in case of embossing, to tens of hundreds of microns as it would be in case of the lamination processes. The flow directors can be linear, such that they substantially align with the axis connecting the proximal and distal end of the pouch, wavy, diamond, honeycomb or any other similar shape, or completely random, that may be interconnected with one another in such a way that the gross effect attained by the flow directors is in the desired direction of flow. The flow directors can be on the entire surface or some part of the inner surface of one or both pouch layers.

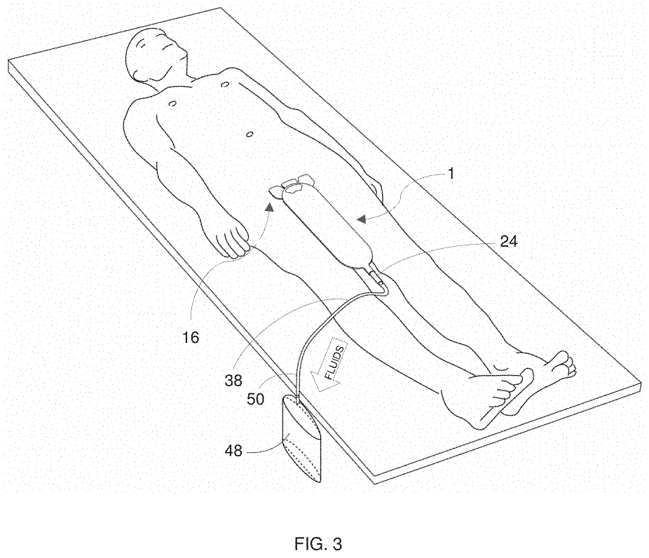

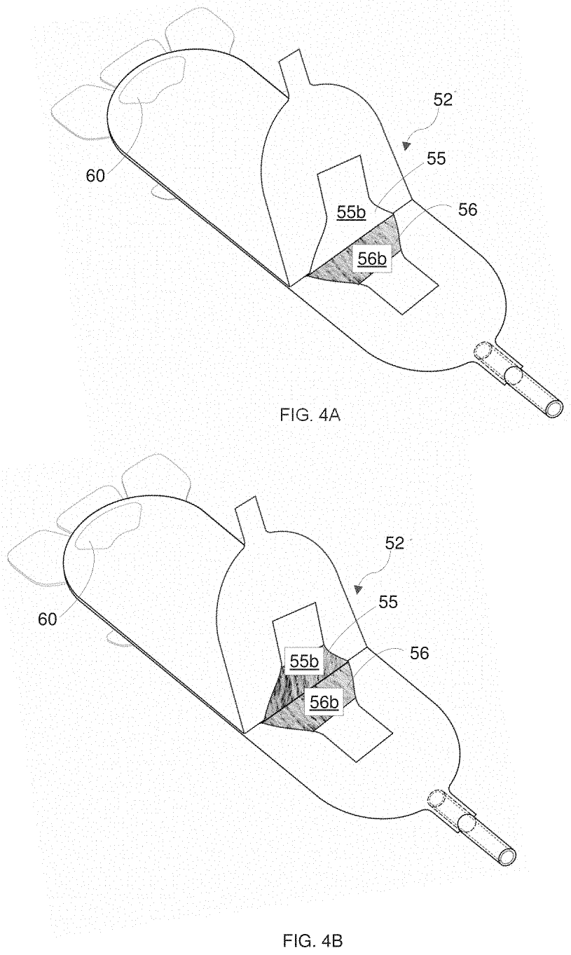

FIGS. 4A-4C show pouch embodiments with differing layups that include surfaces with flow directors. With reference to the cut-open illustration in FIG. 4A, an embodiment of a pouch 52 is shown comprising a first fluid compartment formed from walls 55 and 56, and each having inner surfaces 55b and 56b respectively, and having a fluid transmission passageway leading from the first fluid compartment to a second fluid compartment, and also having an air exchanger 60. In embodiments, the inner surfaces of one or both of walls 55 and 56 may have flow directors which may, for example, be molded, hot-rolled, deposited, bonded, spray-coated, etched, formed or embossed on the inner surface or attached as a laminate on the inner surface. In FIG. 4A, inner surface 56b is shown in a shaded form to illustrate that it has flow directors. Likewise in FIG. 4B, both of inner surfaces 55b and 56b are shown in a shaded form to illustrate that each such inner surface has flow directors.

While FIGS. 4A and 4B illustrate flow directors in the first fluid compartment, it would be understood that in other embodiments, flow directors may be formed within both of the first fluid compartment and the second fluid compartment, and in yet other embodiments, the flow directors may be formed only within the second fluid compartment.

In various embodiments, the flow director layers may be formed on, or laminated onto, or otherwise affixed onto an inner surface of a wall of a fluid compartment, or may not be formed on, or laminated onto, or affixed to an inner wall, and may instead simply be located in an untethered form between two adjoining inner walls of a fluid compartment.

Referring now to FIG. 4C, another embodiment of a pouch 72 is shown which may have a first wall 75, which is connected to a second wall 76. The first wall 75 and second wall 76 may define either of the first fluid compartment or the second fluid compartment within the pouch. This embodiment differs from that in FIG. 4B in that both the inner surface 76b of the second wall 76 and the inner surface 75b of the first wall 75 may be bare, while flow directors may be either molded, hot-rolled, deposited, bonded, spray-coated, etched, formed or embossed or attached as a laminate on one or both sides of a middle layer 79 which is disposed between the second wall 76 and the first wall 75. Furthermore, the middle layer 79 resides captive between the first wall 75 and the second wall 76 and it may be floating, that is not directly attached, or it may be attached to one or both walls 76 or 75 by, for example, lamination, bonding or heat staking. One skilled in the art will recognize that there are many types of flow directors and concomitant manufacturing and attachment methods that serve to separate the walls and provide small channels for fluid to pass, and several examples will be described in more detail below.

In this and other pouch embodiments disclosed herein, (referring to FIGS. 4A and 4C) the first wall 55, 75 and second wall 56, 76 may be attached at their peripheral edges, or at other points between them, or a combination of the peripheral edges and discrete or continuous surfaces or lines between the layers. The walls may be joined by any method for joining polymeric sheets as known by one skilled in the art such as heat sealing (heat staking) or adhesive or solvent bonding. The conduit member retainers may be made by joining discrete spots or lines between the first wall 55, 75 and second wall 56, 76 for example, and may also be locations where the first wall 55, 75 and second wall 56, 76 are joined together. Alternatively, in other embodiments, the conduit members may be joined directly to one or both walls directly as described elsewhere in this disclosure.

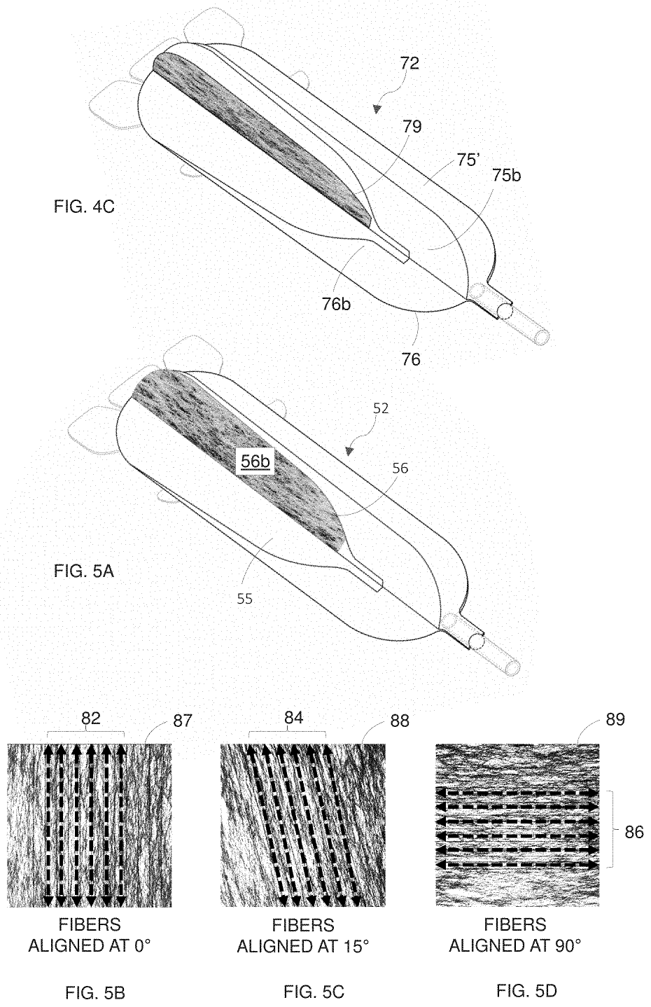

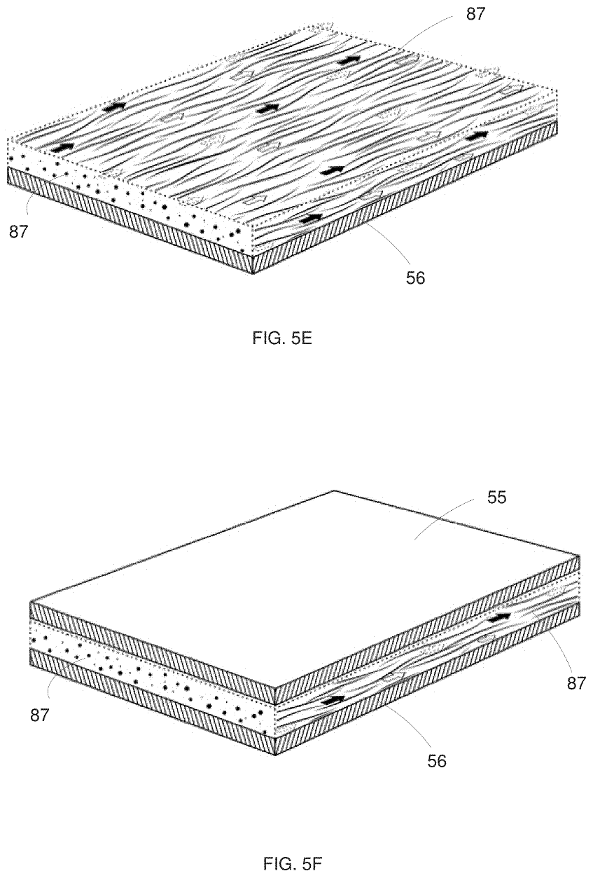

As noted above, flow directors may be located on any one or more of the surfaces on the inside of the pouch. In some embodiments, and with reference to the pouch 72 in FIG. 5A, the inner surface 56b of a second wall 56 may have flow directors as depicted by the shaded striations extending longitudinally in FIG. 5A. This arrangement is essentially the same as that shown in FIG. 4A, except that the second wall 56 is peeled up longitudinally to show a portion of the inside of a fluid compartment of the pouch 52. The flow directors may be any type of texture that tends to keep the second wall 56 and first wall 55 separate in at least some areas of the inside internal compartment of the first fluid compartment or the second fluid compartment of the pouch so that liquid (urine) within such compartment and air may flow without having a partial or total vacuum lock condition—a condition wherein the walls stick together due to the air suction and/or capillary or stiction forces between the walls. In addition, the flow directors may have some preferential orientation, either entirely across the surface, or on average, such that urine is channeled from one end to the other end of the pouch as it is drawn by the tips of the conduit members.

The orientation of the flow directors is further emphasized in FIGS. 5B-5D, which show various orientations of the directors with respect to the longitudinal (proximal to distal) axis of a pouch. FIG. 5B shows a section of a wall within the pouch or within a fluid compartment within the pouch with directors indicated by shaded striations oriented along 0 degree vectors 82 indicating alignment with the pouch longitudinal axis, as described below in more detail. FIG. 5C shows a section of a second wall with directors indicated by shaded striations oriented along vectors 84, indicating 15 degrees of angle with respect to the pouch longitudinal axis. FIG. 5D shows a section of a second wall with directors indicated by shaded striations oriented along 90 degree vectors 86 indicating orthogonality with the pouch longitudinal axis and preferred air/liquid flow direction. Depending on the structural features used to create the flow directors, they may or may not be fully aligned homogenously. That is, a distribution of channels, fibers, or whiskers may have a net orientation along the 0 degree vector 82 orientation, but there may be a distribution of angles due to manufacturing processes such that the average angle of these features aligns approximately with the vector 82 orientation (approximately 0 deg.).

In some embodiments, fibers 87, 88, and 89, as shown in FIGS. 5B-5D, may serve as flow directors, and they may be attached to the wall of any of the layers described above and shown in FIGS. 4A-4C, or the fibers 87, 88, and 89 may be attached to a substrate that is, in turn, attached to one of the pouch walls. The fibers 87 may be continuous along the length of the pouch or discrete, having many smaller lengths that overlap, and the size may be lightweight, such as 1-10 denier, or heavy weight in some applications, up to 50 denier or heavier. The height of the fibers 87 should be high enough to allow the fibers 87 to create a channel for fluids to flow between the fibers 87 as the pouch walls are brought together by force or under suction. That is, the height should be large enough to allow urine to flow between the pouch walls without incurring a vacuum lock.

FIG. 5E shows a detailed view of a section of a wall 56 of a pouch having flow directors. In this example, the flow directors are comprised of fibers 87 indicated by the various sized dots shown on the end of the cross-section and lines shown on the top and side surfaces. The fibers 87 are generally aligned such that fluid flows in the direction of the arrows, which may be aligned from the proximal to distal end of the pouch. Gaps are formed between the fibers 87 which create grooves for fluid flow. For example, as shown in FIG. 5F, when a first wall 55 and an opposed second wall 56 of a pouch are drawn together, by vacuum for example, the fibers maintain flow channels so that fluid may flow as indicated by the arrows, the fluid being drawn, for example, by a pressure differential acting on the fluid in that direction.

In some embodiments, the manufacturing process to create the substrate involves having fibers 87 laid down along a moving conveyor belt and then blown with hot air to melt them together. This substrate may be described as melt-blown, airlaid, or hot air through. The fibers may be made of a single material, or multiple materials, for example, wherein two materials (e.g., PE and PP) are joined together in small extruders such that each strand has both materials in a base/binder configuration, wherein the binder has a lower melting temperature. The base/binder ratio may be, for example, from 1:20 up to 20:1. The final substrate (sheet) that contains the fibers may have a sheet weight of about 5-500 gsm and a thickness of approximately 5-500 microns, in some example embodiments.

The fiber material may be a thermoplastic polymer, a thermoset polymer, or combination thereof, or a natural fiber. Examples of suitable thermoplastics include but are not limited to: polyethylene, polypropylene, polyethylene terephthalate, polyamide, polyvinyl chloride, polyester, polyether, polyurethane, polytetrafluoroethylene, block-copolymer elastomers, polyamide. Examples of suitable thermosets/rubbers include but are not limited to: butyl, chloroprene, epichlorohydrin, ethylene/acrylic, ethylene-propylene, fluorocarbon, fluorosilicone, silicone rubber, natural rubber, nitrile, hydrogenated nitrile, perfluoroelastomer, polyacrylate, polysulfide, styrene butadiene. Examples of suitable natural fibers include but are not limited to: linen, silk, and wool.

In some embodiments, the fibers 87 are hydrophobic either because the underlying fiber material is inherently hydrophobic, or it is treated with a hydrophobic compound making it non-wicking. Hydrophobicity tends to prevent attraction (wetting or wicking) of the urine to the surface. However, depending on the nature, size, and orientation of the fibers, a neutral or hydrophilic material may be similarly functional. Additionally or alternatively, the fibers may be constructed of a nonwoven material.

A nonwoven fibrous surface may be particularly slippery against skin so that the penis does not get stuck while inserting it into the pouch, making the application of the device convenient for the operator and comfortable for the patient. Furthermore, a nonwoven fibrous material tends to dry to the touch rapidly when the urine is partially or fully evacuated so that nearly all liquid can be removed quickly, reducing the risk of skin maceration.

One skilled in the art would recognize that there are many physical shapes or materials that can maintain space between the pouch walls to prevent vacuum locking. By way of example, a pouch may have a plurality of elongate members, such as rods, located inside the pouch, between a first wall and a second wall. The elongate members may be discrete or attached to each other, and they may be captive inside of the pouch with or without a direct attachment. Alternatively, they may be attached to either the first wall or the second wall, or both by bonding or melting with heat (e.g. heat staking). The elongate members are approximately aligned with the longitudinal axis of the pouch and they may span the entire length of the pouch or have a length shorter than the pouch.

In other embodiments, a pouch may have corrugated sheet or a mesh or a ribcage or a lattice located inside of the pouch between a first wall and a second wall. The corrugated sheet or mesh or ribcage or lattice may be captive inside of the pouch with or without a direct attachment. Alternatively, the sheet or mesh or ribcage or lattice may be attached to either the first wall or the second wall, or both by, for example, bonding or melting with heat (e.g., heat staking or thermal bonding). Channels formed within a corrugated sheet or grids formed in the mesh or ribcage or lattice may be aligned or approximately aligned with the longitudinal axis of the pouch, and they may span the entire length of the pouch or have a length shorter than the pouch.

Flow directors, including the aforementioned flow directors disclosed herein, or other embodiments of directors not particularly detailed here but within the scope of the disclosure, may be made of a polymeric material that may be fabricated by injection molding, blow molding, layer deposition, extruding, or compression molding, for example. Examples of suitable thermoplastics include but are not limited to: polyethylene, polypropylene, polyethylene terephthalate, polyamide, polyvinyl chloride, polyester, polyether, polyurethane, polytetrafluoroethylene, block-copolymer elastomers, and polyamide. Examples of suitable thermosets/rubbers include but are not limited to: butyl, chloroprene, epichlorohydrin, ethylene/acrylic, ethylene-propylene, fluorocarbon, fluorosilicone, silicone rubber, natural rubber, nitrile, hydrogenated nitrile, perfluoroelastomer, polyacrylate, polysulfide, styrene butadiene. Alternatively, the flow directors may be made of a metal such as steel, nitinol or any other suitable metal. The flow directors disclosed herein may be hydrophobic either due to the innate characteristics of the material or due to a hydrophobic coating applied to the material. A hydrophobic surface tends to be non-wicking so as to prevent urine from adhering to the surface so that it can be more readily entrained in the vacuum-driven flow of fluid through the pouch.

In some embodiments, flow directors may be embedded in a first wall or a second wall, or in a middle layer within the pouch or within either or both of the first fluid compartment and the second fluid compartment. The features may be embossed or etched into the material or molded or extruded as part of the fabrication process for the sheet or film material or substrate having the texture.

One skilled in the art would recognize that there are many raised patterns that can be made on the inside of one or more pouch surfaces to create an effective flow director. Examples include straight lines, grids, and repetitive patterns such as herringbone, fish scales, diamond, picket, triangle, or arabesque. In one embodiment the pouch may have protuberances arranged on inner surfaces of one or more walls of the first fluid compartment or the second fluid compartment. The protuberances on an inner surface of a wall may contact the opposing wall to prevent the walls from coming into full contact over significant areas, thus reducing the incidence of vacuum lock. Therefore, gaps between the protuberances create interstices (acting as flow directors) through which air and urine can flow. The protuberances may be actual solid or semi-solid bumps on the inner walls that are molded in place, or attached via melting, or bonded in place. In other embodiments, the protuberances may be formed in the wall, like a dome, having the same wall thickness as the nominal wall.

Flow directors need not be uniformly arranged on the entire inner surfaces of the pouch or of the first fluid compartment or the second fluid compartment. In some embodiments, flow director structures may simply be formed or provided on inner surfaces of the pouch or of the first or second fluid compartment that are located in the vicinity of one or more fluid inlets within the pouch or within the first or second fluid compartment.

The pouches disclosed herein may have walls that are made of any suitable material that is highly flexible, i.e., low in flexural modulus and/or thin-walled. In addition, the pouch material may be chosen in some embodiments so that the resulting wall has a thickness of about 5-100 μm, or in some embodiments the thickness may be up to 1 mm. The pouch is liquid impermeable and may be heat weldable, or may otherwise be laminated, bonded, or solvent bonded, to another flexible wall. Examples of suitable materials may include but are not limited to, polyurethane, ethylene vinyl acetate, polyethylene, silicone, rubber, latex, polyolefin, or any other suitable thermoplastic. Furthermore, the pouch walls may be made fully or partially of an odor-blocking material such as a polyamide, polyvinylidene dichloride, ethylene vinyl alcohol or EVOH or similar materials, or alternatively, the odor-blocking material may comprise one or more films coextruded with other suitable materials or otherwise attached to the pouch walls.

While the comfort and environment inside of the pouch is of paramount importance, as described above, the external surface also touches the body on and in-between the legs; although this surface is not typically wet, it may cause a sensation or irritation to the skin over time. As such, the outer surface(s) of the pouch may have a layer of material attached to aid in patient comfort. The layer may be made of suitable thermoplastics, including but not limited to: polyethylene, polypropylene, polyethylene terephthalate, polyamide, polyvinyl chloride, polyester, polyether, polyurethane, polytetrafluoroethylene, block-copolymer elastomers, silicone, rubber, latex, or polyamides. Alternatively, or in combination, natural fibers may be used, including but not limited to cellulose-based materials such as cotton, linen, rayon, or various others such as silk or wool.

### Fluid Flow System

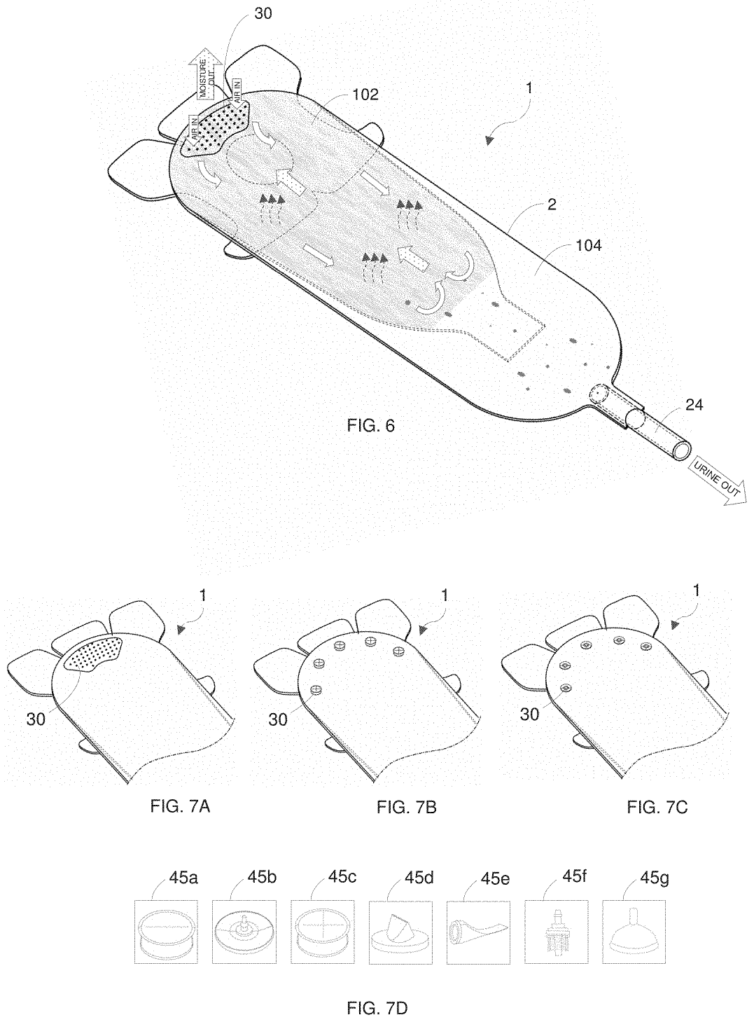

The air and urine flow pathways are illustrated schematically in FIG. 6 in the context of the urine removal device 1 described previously herein. FIG. 6 shows a pouch 2 comprising a first fluid compartment 102 and a second fluid compartment 104, without urine, with air flow paths depicted by white arrows. In embodiments disclosed herein, the air flow into the pouch is enabled through air exchanger(s) 30 The air is entrained in the pouch 2 through air exchanger(s) 30, after which it flows from the proximal end of the pouch towards the distal end of the pouch, and more specifically from first fluid compartment 102 and into second fluid compartment 104. Urine within the pouch 2 is drawn out of, or expelled through outlet conduit member 24, while, the air can circulate out of pouch 2 through air exchanger(s) 30. Notably, in embodiments such as this, the air exchanger(s) 30 are located near the proximal end of the device 1. This allows the air flow to pass by the penis (not shown) because the aperture through which the penis is inserted may be located distal to the air exchanger(s) 30.

FIG. 7A illustrates a device 1 wherein air exchanger(s) 30 comprises a fabric or film that allows air to enter or exit but does not allow liquid permeability. FIG. 7B illustrates a device 1 wherein air exchanger(s) 30 comprises one or more one-way slit valves that are air permeable but which stick together or seal shut when in contact with moisture. FIG. 7C illustrates a device 1 wherein air exchanger(s) 30 comprises one or more one-way dome valves that are air permeable but which stick together or seals shut when in contact with moisture. FIG. 7D illustrate various embodiments (for example a slit valve 45a, an umbrella or flapper valve 45b, a cross-slit valve 45c, a duckbill valve 45d, a flutter valve 45e, a check valve 45f, and a dome valve 45g are shown in FIG. 7D as examples) of valve arrangements that can be used for the purposes of implementing the embodiments illustrated in FIGS. 7B and 7C.

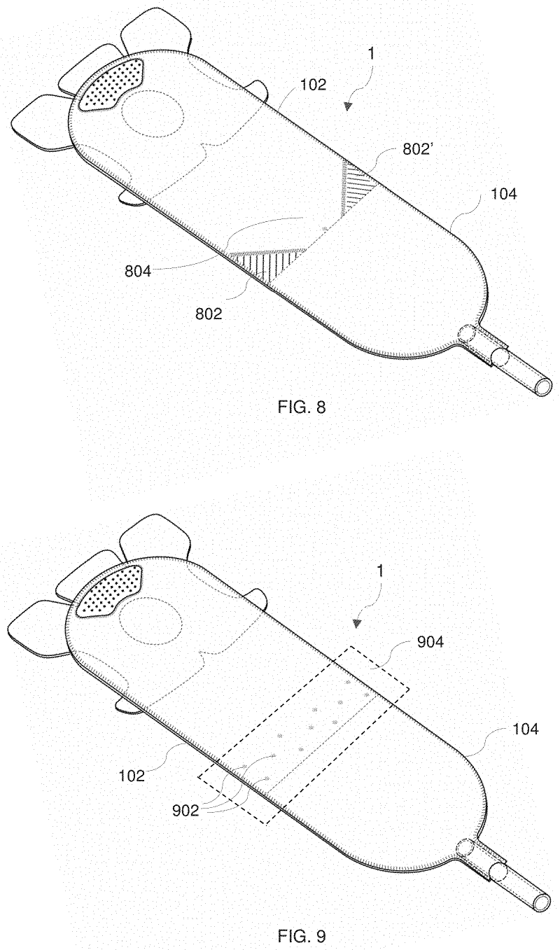

It would be understood that the fluid transmission passageway between the first fluid compartment 102 and the second fluid compartment 104 can be implemented in any number of different ways. For example, as shown in FIG. 8, the first and second fluid compartments 102, 104 may be formed within pouch 2 simply by creating a fluid constriction region 804 formed by sealing together the internal walls of pouch 2 at sealed regions 802, 802′ to create a narrowed passageway to enable fluid to travel from first fluid compartment 102 to second fluid compartment 104—which creates a fluid passageway having a restricted width between the two fluid compartments. In another embodiment shown in FIG. 9, the fluid transmission passageway may be formed by sealing opposite internal walls of the pouch 2 at a region 904 between the first and second fluid compartments 102, 104, in a stippled or dotted sealing arrangement comprising a plurality of point seals or point welds 902 between opposite internal walls of pouch 2—thereby defining a fluid constriction region 904 formed between the first fluid compartment 102 and the second fluid compartment 104—which reduces the available volume of the fluid passageway between the two fluid compartments 102, 104— and which serves to prevent fluid reflux from the second fluid compartment 104 into first fluid compartment 102.

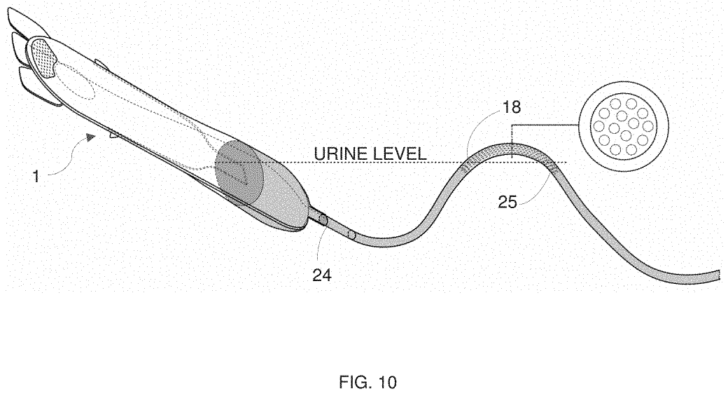

FIG. 10 shows an embodiment of device 1, wherein a drain tube 25 connecting an outlet conduit member 24 and a urine collection receptacle (not shown) is provided with a plurality of non-absorbing strands 18—which form a capillary structure that draws urine through the tube 25 and in the direction of the urine collection receptacle through capillary action, and thereby facilitates removal of urine from pouch 2 and into the urine collection receptacle. As a result of this capillary structure, urine is drawn out of the second fluid compartment of device 1 and into a urine collection receptacle despite orientations where portions of drain tube 25 travel in an upward sloping direction in the direction from device 1 towards the urine collection receptacle.

FIGS. 11A to 11F illustrate an embodiment of device 1, wherein outlet conduit member 24 is provided with a bell siphon assembly 242 configured to ensure that fluid passing into the second fluid compartment from the first fluid compartment is pushed out of outlet conduit member 24 by action of the bell siphon assembly 242.

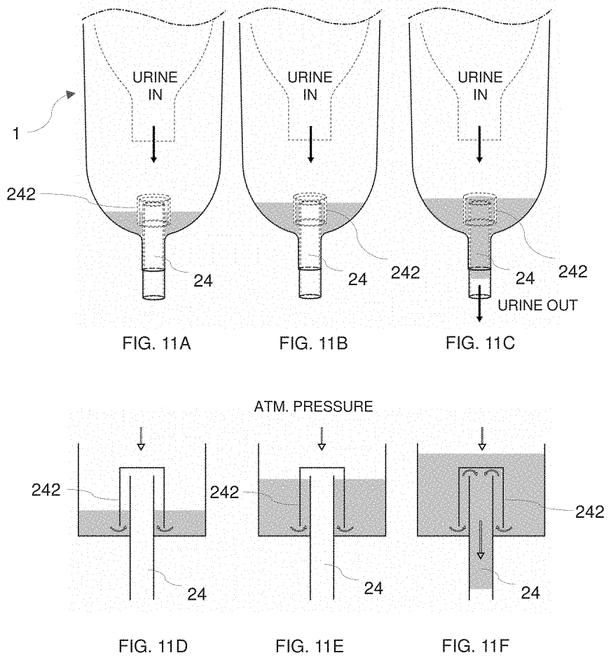

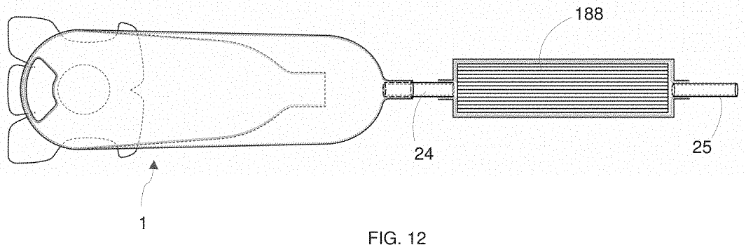

FIG. 12 shows an embodiment of device 1, wherein an open-cell foam assembly 188 is provided as an interconnection between an outlet conduit member 24 and a drain tube 25 that provides fluid passageway into a urine collection receptacle (not shown). The open-cell foam assembly 188 is provided with a plurality of fluid channels defined within the foam assembly, each of which interconnect outlet conduit member 24 and tube 25—such that if foam assembly 188 is positioned under a patient's limb, the pressure from the limb will at worst, only partially occlude the fluid passageways between outlet conduit member 24 and tube 25, and at least some fluid channels within foam assembly 188 will continue to provide fluid passageway between device 1 and a urine collection receptacle. Additionally, use of a foam assembly 188 ensures that patient discomfort is minimized even if the foam assembly is trapped beneath the patient's limb.

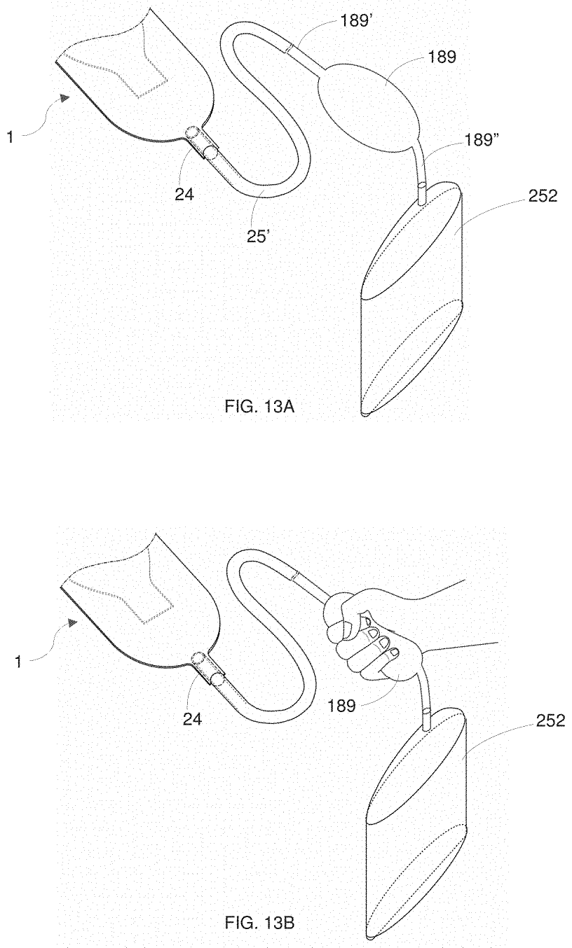

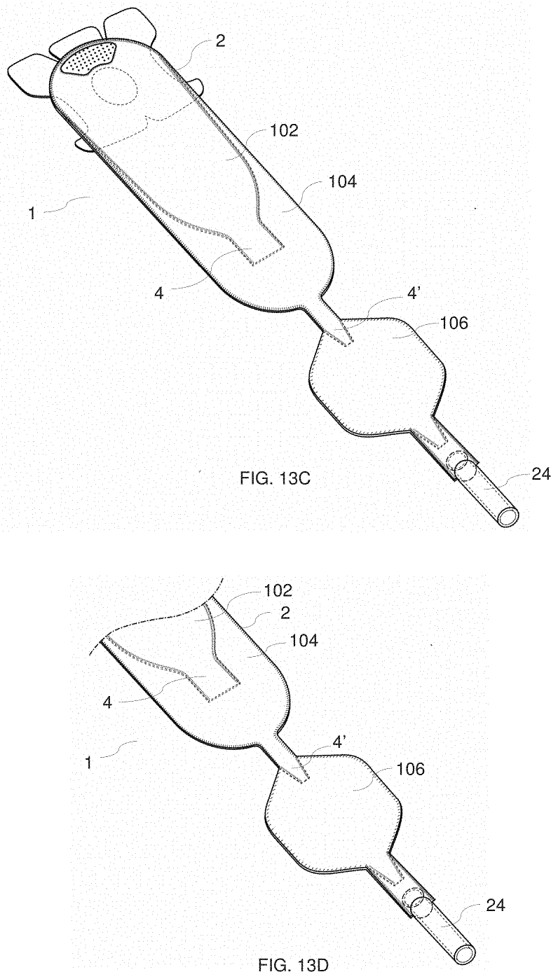

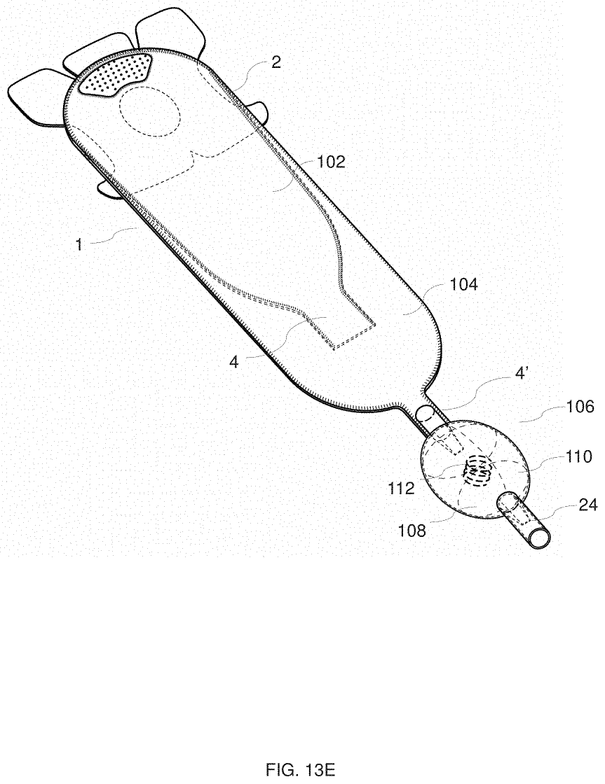

FIGS. 13A and 13B show an embodiment of device 1, wherein a suction bulb 189 is provided as an interconnection between an outlet conduit member 24 and a urine collection receptacle 252. The suction bulb 189 is located such that a tube 25′ connects outlet conduit member 24 to a fluid inlet 189′ of suction bulb 189, and a fluid outlet 189″ of suction bulb 189 is connected to an inlet of urine collection receptacle 252. Squeezing the suction bulb 189 creates negative pressure in the second fluid compartment of device 1 and draws urine that has collected in the second fluid compartment out of device 1, through suction bulb 189 and into urine collection receptacle 252.

FIGS. 13C and 13D show another embodiment of device 1, wherein a third fluid compartment 106 is provided as an intermediate fluid compartment between second fluid compartment 104 and outlet conduit member 24. In this embodiment, the pouch 2 comprises a first fluid compartment 102, a second fluid compartment 104 and a first fluid transmission passageway 4 connecting the first fluid compartment 102 and the second fluid compartment 104—wherein the first fluid transmission passageway 4 comprises a first flutter valve (or a first thin-film unidirectional fluid flow valve) configured to enable for fluid flow from the first fluid compartment 102 to the second fluid compartment 104, and to prevent fluid flow from the second fluid compartment 104 to the first fluid compartment 102. The first fluid transmission passageway 4 comprises a fluid inlet, a fluid outlet and a lumen that is formed from one or more collapsible flexible sheets. The fluid inlet of the first fluid transmission passageway 4 is provided towards or at a distal end of the first fluid compartment 102. The fluid outlet of the fluid transmission passageway 4 is provided within the second fluid compartment 104. The lumen connects the fluid inlet and the fluid outlet and forms a first fluid passageway 4 therebetween. In an embodiment, the first fluid transmission passageway 4 is sized to enable fluid flow from the first fluid compartment 102 to the second fluid compartment 104 at a flow rate of at least 10 ml per min. The first fluid compartment 102 is positioned relatively closer to the proximal end of the pouch 2 and relatively further from the distal end of the pouch 2, whereas the second fluid compartment 104 is positioned relatively closer to the distal end of the pouch 2 and relatively further from the proximal end of the pouch 2. The pouch 2 has an aperture sized to permit a male patient's penis to enter the pouch within the first fluid compartment.

Additionally, pouch 2 has a third fluid compartment 106 positioned as an intermediate fluid compartment between the second fluid compartment 104 and an outlet conduit member 24, and a second fluid transmission passageway 4 ‘ connects the second fluid compartment 104 and the third fluid compartment 106—wherein the second fluid transmission passageway 4’ comprises a second flutter valve (or a second thin-film unidirectional fluid flow valve) configured to enable fluid flow from the second fluid compartment 104 to the third fluid compartment 106, and to prevent reflux of fluid from the third fluid compartment 106 back into the second fluid compartment 104. The second fluid transmission passageway 4′ comprises a fluid inlet, a fluid outlet and a lumen that is formed from one or more collapsible flexible sheets. The fluid inlet of the second fluid transmission passageway 4′ is provided towards or at a distal end of the second fluid compartment 104. The fluid outlet of the second fluid transmission passageway 4′ is provided within the third fluid compartment 106. The lumen connects the fluid inlet and the fluid outlet and forms a second fluid passageway 4′ therebetween. Outlet conduit member 24 is provided at a distal end of third fluid compartment 106 so that urine that is delivered into the third fluid compartment 106 from the second fluid compartment 104 can flow out and into a urine collection receptacle (not shown) that is connected with outlet conduit member 24.

It has been found that by providing a third fluid compartment 106 connected to the second fluid compartment by a second fluid passageway 4′ that comprises a second flutter valve, a pressure based siphon arrangement can be set up to force urine from pouch 2 into a urine receptacle connected to outlet conduit member 24. Urine that is released by a patient into a first fluid compartment 102 passes through the first fluid passageway 4 into the second fluid compartment 104 and thereafter passes through the second fluid passageway 4′ into the third fluid compartment 106. By squeezing or applying manual pressure on the external walls of third fluid compartment 106, fluid collected in the third fluid compartment 106 is forced onward through outlet conduit member 24 and in the direction of a urine receptacle (not shown). Additionally, the flow of urine from the third fluid compartment 106 and out of outlet conduit member 24 creates negative pressure within pouch 2, and/or first, second and third fluid compartments 102, 104, 106— such that fluid in the first and second fluid compartments 102, 104 are drawn out of said compartments, into the third fluid compartment 106 and are expelled out of outlet conduit member 24. Additionally, by forming the second fluid passageway 4′ as a second flutter valve, the second fluid passageway 4′ reduces or eliminates reflux of any urine back from the third fluid compartment 106 in the reverse direction towards the first fluid compartment 102—despite application of any external pressure or force to the third fluid compartment 104 for establishing pressure to expel urine from pouch 2.

In an embodiment, the first fluid transmission passageway 4 is sized to enable fluid flow from the first fluid compartment 102 to the second fluid compartment 104 at a flow rate of at least 10 ml per min. In an embodiment, the second fluid transmission passageway 4′ is sized to enable fluid flow from the second fluid compartment 104 to the third fluid compartment 106 at a flow rate of at least 5 ml per min.

The pouch 2 and each of the fluid compartments 102, 104, 106 may be formed from a plurality of collapsible flexible sheets that are affixed together or sealed together along the peripheries of the pouch and/or first or second fluid compartments. The flexible sheets may comprise one or more fluid-tight (liquid-impermeable) materials (for example, polyvinyl chloride, polypropylene, polyethylene, low-density polyethylene, high-density polyethylene, ethylene-vinyl acetate, polyvinylidene dichloride, biaxially oriented polypropylene, ethylene vinyl alcohol, natural rubber latex, silicon rubber, polyurethane, coated synthetic or natural fabric, etc.) wherein the fluid-tight materials define internal compartments for the pouch and the first and second fluid compartments therewithin. The fluid-tight materials form one or more external surfaces and internal surfaces for the pouch. The aperture in first fluid compartment 102 may be fixedly or adaptably sized to enable a male patient's penis to be inserted therethrough and to be positioned within the first fluid compartment. Additionally, each of the pouch 2, first fluid compartment 102 and aperture are configured such that upon insertion of the patient's penis, at least a part of the penis (for example, at least the tip of the penis or the meatus) and optionally, substantially the whole of the penis, is housed within the first fluid compartment 102 in the region between the aperture and the second fluid compartment, and/or between the aperture and the distal end of the pouch.

FIG. 13E illustrates a more particular embodiment of the urine removal device illustrated in FIGS. 13C and 13D, wherein the third fluid compartment 106 is held or housed between a pair of rigid or semi-rigid pressure application surfaces 110, wherein the pair of rigid or semi-rigid pressure application surfaces 110 can be squeezed together to apply pressure on the third fluid compartment 106—causing fluid within the third fluid compartment 106 to be expelled from said compartment 106 and towards the urine collection receptacle (not shown) that is connected with outlet conduit member 24. In a specific embodiment, the pair of rigid or semi-rigid pressure application surfaces are separated by a resilient member 112 (for example a spring) that urges each of the pair of rigid or semi-rigid pressure application surfaces 110 away from each other. Resilient member 112 is compressed when pressure application surfaces 110 are squeezed together, and when the squeezing force ceases to be applied, resilient member 112 forces the two pressure application surfaces 110 apart from each other. A patient or health care provider can intermittently squeeze the pressure application surfaces 110 to ensure that urine within the third fluid compartment 106 is periodically expelled from third fluid compartment 106 and into the urine collection receptacle. Providing rigid or semi rigid surfaces which can be used to squeeze the third fluid compartment 106 provides for an improved patient and/or health care provider user experience, and also enables more efficient expulsion of fluid from third fluid compartment 106, when compared to the flexible walls of the third fluid compartment 106 being squeezed directly by a patient or health care provider's hand or fingers.

While the embodiment described in connection with FIG. 13E shows the pair of rigid or semi-rigid pressure application surfaces 110 disposed on either side of the third fluid compartment 106, a pair of rigid or semi-rigid pressure application surfaces may alternatively or additionally be disposed on either side of the second fluid compartment 104 such that application of pressure to squeeze this pair of rigid or semi-rigid pressure application surfaces results in urine being expelled out of the second fluid compartment 104.

FIG. 14A illustrates a problem observed in device 1, wherein kinking or wrinkling of the distal end of device 1 (or of the second fluid compartment) can result in occlusion of the fluid passageway leading to outlet conduit member 24—and in prevention of fluid flow out of device 1. As shown in FIGS. 14B and 14C, positioning a cup shaped funnel or cup shaped porous member at a distal end of device 1 (or at a distal end of the second fluid compartment), between the first fluid compartment and the outlet conduit member 24 inhibits wrinkling or kinking and ensures that a fluid passageway is maintained in partially or wholly unoccluded form so that urine can pass through from the second fluid compartment and out of outlet conduit member 24. BODY ATTACHMENT

In the embodiments disclosed, the urine removal device may be attached at the base of the penile shaft, scrotum, and abdomen. There are several advantages and challenges of having an interface that is affixed on the groin, pelvic and abdominal region. In addition to the shape of the adhesive, the shape of the orifice, method of application (different peel points), and material of the adhesive may be tailored to this area of anatomy.

Due to a combination of factors, external collection systems face challenges with patient attachment as well as dislodgement. Variation in patient anatomy creates challenges in a one-size-fits-all solution. Genital skin is sensitive and prone to pain, which limits the strength of adhesive able to be used. The abdominal and genital region may also have various creases and folds that are unique to each patient. Movement of legs and hips causes significant bending, elongation, and compression of genital and perineal skin, requiring unique functions of both the adhesive and connected materials. Lastly, temporary containment of urine inside of an enclosed space increases weight, and subsequent force, acting on the adhesive, exacerbating the risk of dislodgement.

An embodiment of an adhesive patch 410 is illustrated in FIG. 15A. The adhesive patch 410 has an aperture 415 suitable to fit over the shaft of a penis and a wide proximal end 412 and a wide distal end 414, which attach to the lower abdomen and scrotum respectively. In embodiments, the adhesive patch 410 may have a narrowed central section 424 defined by concave sidewalls 420 as shown in FIG. 15A. The wide proximal end 412 provides a large surface area to improve adhesion to the body while stabilizing against twisting motions where it attaches to the suprapubic region. The width of the proximal end 412 may be approximately 15 cm across or between 2 and 30 cm across in embodiments. The proximal end 412 may have one or more slits 416 dividing the adhesive patch 410 into proximal tabs 417. Likewise, the distal end may have at least one slit 418 dividing the adhesive patch 410 into distal tabs 419. The slits 416 and 418 can reduce tension or compression of the adhesive patch 410 during body movement by providing a relief between each section of attached skin that is deforming, thus allowing the adhesive patch 410 to deform with the body without excessively resisting the motion which can cause discomfort or lead to dislodgement. The slits 416 and 418 may be straight cuts or, as shown, they may be approximately “v” shaped or any other shape, and in some embodiments having rounded corners to be more gentle and atraumatic to the skin.

The distal end 414 may be narrower than the proximal end 412 so that it fits onto the scrotum. The width of the distal end 414 may be approximately 14 cm across or, for example, from 2 cm to 30 cm in some embodiments.

The proximal end 412 and the distal end 414 attach to the suprapubic region and scrotum respectively, while the central section 424 lays around the penile shaft. As such, the central section 424 is narrower than the proximal end 412 and distal end 414 of the adhesive patch 410. The central section 424 may be approximately 9 cm across or from 5 cm to about 20 cm in some embodiments. The topology consists of rather variable surfaces in the anatomy in the region of the central section 424 so the reduced width can lessen the contact, and therefore the forces transferred, due to movement of, for example, the legs or scrotum, which can reduce the incidence of dislodgment of the adhesive patch 410. Furthermore, when the adhesive patch is applied around the penile shaft, the operator may press the large surface areas of the adhesive patch 410 onto the large surface areas of the body, for example by pressing the wide proximal end 412 onto the suprapubic region and the somewhat less wide distal end 414 onto the scrotum, while the central section 424 seats down around the shaft of the penis. The narrow, concave shape of the central section 424 facilitates this section seating deep enough to seat against the skin adjacent to the shaft of the penis without hanging up or being held back by the adjacent skin which has a relatively topology in the pubic region.

FIG. 15B shows the adhesive patch 410 with the layers separated on a corner to more clearly show the different layers in this embodiment. The substrate layer 421 is a fixed layer in that it is permanently attached to the pouch on its outer surface 421a and it is attached to the adhesive layer 422 on its inner surface 421b. The adhesive layer 422 may comprise a single adhesive layer or a layup of a plurality of layers configured to conform with skin on one side and the substrate layer 421 on the other side. The substrate layer 421 may be coupled to the pouch by thermal welding (heat staking), bonding with solvents or adhesives, or any other method for attaching thin layers. The substrate layer 421 is generally provided coupled to the pouch so that the adhesive patch 410 is already in place, but, in some embodiments, it may be provided uncoupled so that the operator attaches it to the pouch before use. The substrate layer 421 may be made of any material that allows it to adhere to both the adhesive layer 422 and the pouch; examples include but are not limited to a single or a mixture of a natural or thermoplastic or thermoset polymer in sheet, film, woven or non-woven fabric form; example materials include polyethylene, polypropylene, thermoplastic elastomer (TPE), polyurethane, EVA (ethylene-vinyl acetate), nylon, rayon, etc. In some embodiments, the substrate layer 421 may have a peel strength (relative to the adhesive layer 422) greater than 0.1N/cm.

The adhesive layer 422 is sandwiched between the substrate layer 421 and the release liner 423. The inner surface 422b (not shown) of the adhesive layer 422 is suitable for attachment to the body in the areas surrounding the penis including the scrotum, groin, and suprapubic area. The adhesive layer 422 is amenable to adhesion and removal from skin even with hairs emanating from the skin, while being flexible enough to move with the skin without peeling off. The peel strength with respect to steel can be approximately 0.1-5 N/cm in some embodiments. One skilled in the art will recognize that there many candidate materials that will adhere to the skin for the duration of urine capturing, adhere to the substrate layer 421, be easily removable without excessively pulling on the skin and hair, and leave behind little or no residue on the skin. For example, porous or nonporous silicone adhesives may be particularly suitable as they are comfortable to the patient and may leave no perceptible residue. Other candidate materials include pressure-sensitive adhesives, namely a variety of rubber-based materials, gel-matrix type adhesives like hydrocolloids and hydrogels, and thermoplastic-based adhesives including polyurethanes and acrylics as well as natural adhesive obtained from various plants or animals.

The release liner 423 covers and protects the adhesive layer 422 before use, that is, during manufacturing, shipping, and handling. The release liner 423 should be easy to release from the adhesive layer 422 so that the adhesive does not stretch and rebound when the operator peels the layers apart, as this may cause the adhesive patch 422 to fold and stick to itself. The release liner 423 should have a peel strength away from the adhesive that is less than both the peel strength between the adhesive layer 422 and the substrate layer 421, and less than the peel strength between the substrate layer 421 and the pouch. One skilled in the art will recognize that there are many candidate materials that are suitable to protect the adhesive layer 422 in such a way, such as, for example, paper-based liners including different combinations of coated and densified kraft papers and laminated papers, or film-based liners such as high-density polyethylene and polyester thermoplastics. Additionally, the use of release agents along with the release liners may be used.

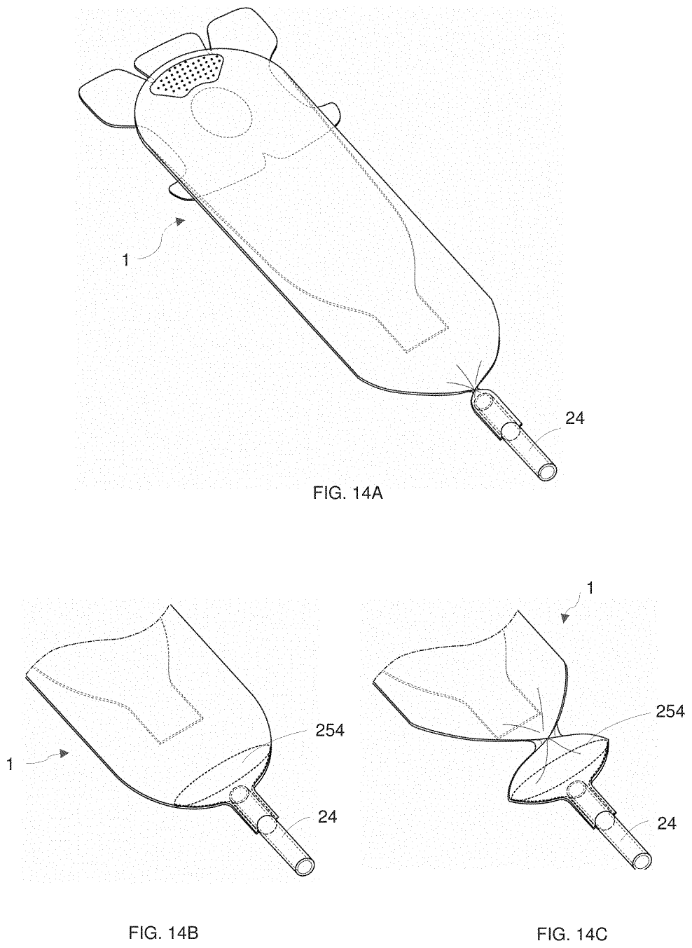

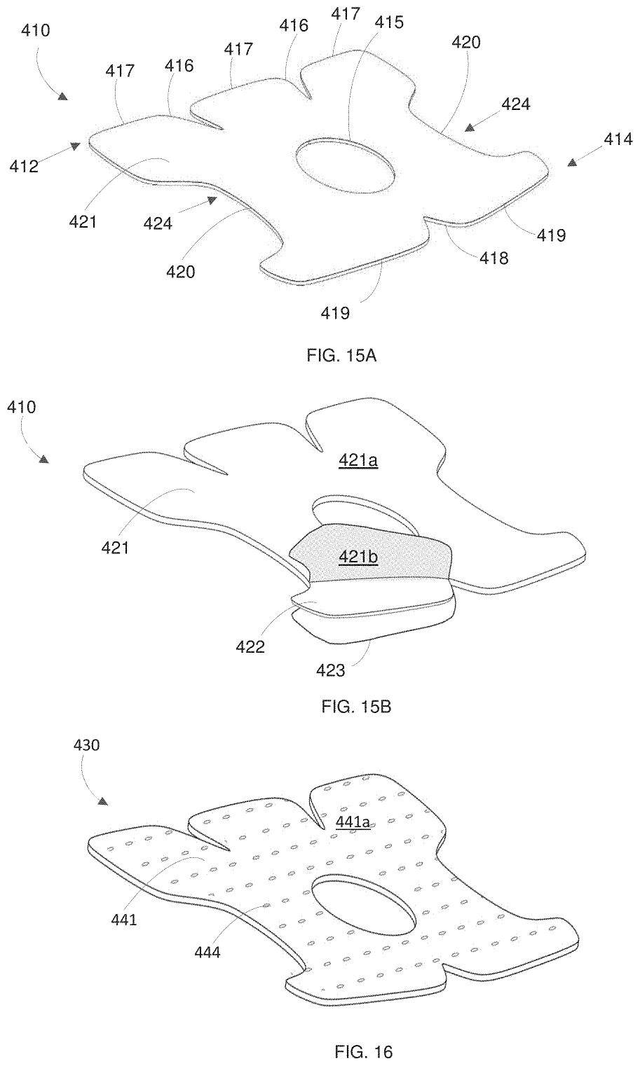

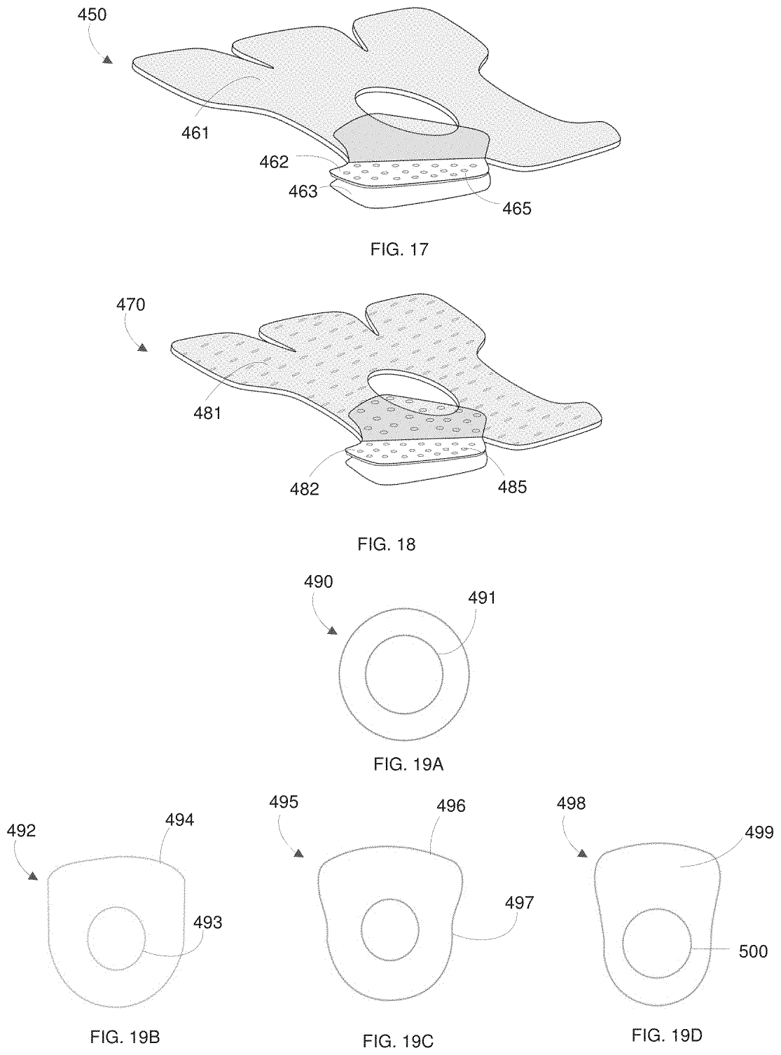

One or more of the layers in the adhesive patch may be perforated to allow sweat to evaporate, which tends to reduce skin maceration. With reference to FIG. 16, an adhesive patch 430 is shown having a substrate layer 441 with perforations 444 shown in the outer surface 441a; in this and the following figures, while the reference numeral points to one perforation, it pertains to the entire array of perforations shown. The perforations 444 may channel through the entire thickness of the substrate layer 441. Alternatively, only the adhesive layer 462 may be perforated 465, as shown in FIG. 17, for improving sweat evaporation. In this adhesive patch 450, the substrate layer 461 is not perforated and the release liner 463 need not be perforated. Finally, as shown in FIG. 18, the adhesive patch 470 both the substrate layer 481 and the adhesive layer 482 have perforations 485 to further enhance sweat evaporation from the skin.

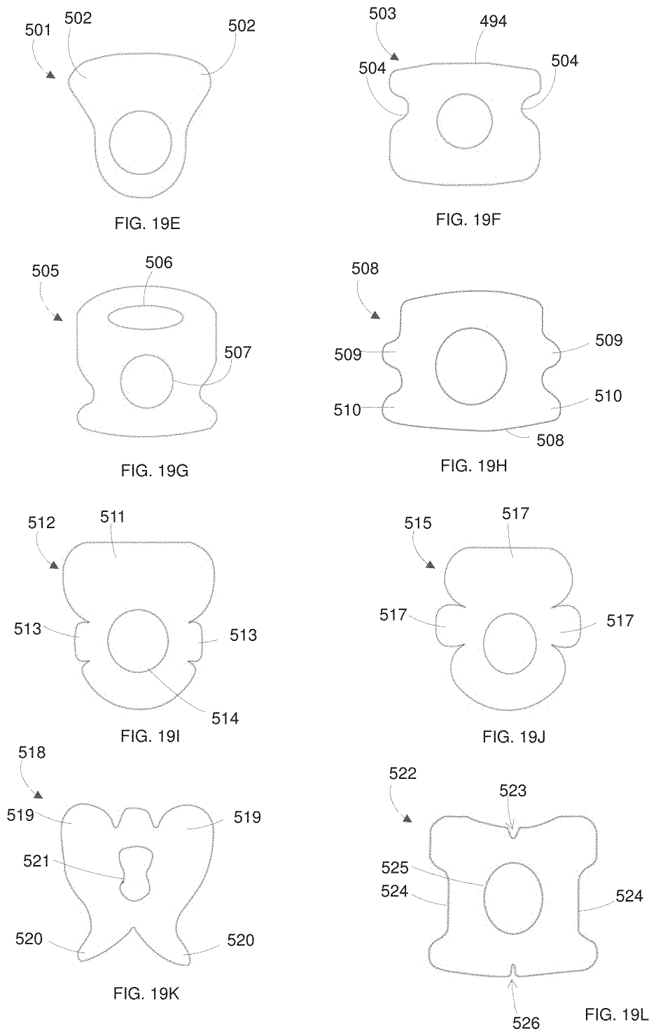

One skilled in the art will recognize that there are many different shapes of the adhesive patch that can be effective to fit within the anatomy around the penis and attach to the suprapubic area and the scrotum while not excessively interacting with the legs and groin area, in what is a geometrically complex area of the male anatomy. FIGS. 19A-L illustrate several embodiments having various shapes as nonlimiting examples; in the examples, the aperture identified designates the region through which the penis is placed for entry into the pouch and proximal is upward on each figure. FIG. 19A shows an adhesive patch 490 that is circular and centered around an aperture 491. FIG. 19B shows an adhesive patch 492 having a proximal section 494 that is high and broad and a distal section that is round, while the adhesive patch 495 of FIG. 19C similarly has a proximal section 496 that is high and broad but has scalloped sides 497. The adhesive patch 498 shown in FIG. 19D has a shape that is narrow around the aperture 500 with a high proximal section 499 opposed by a round distal end. The adhesive patch 501 shown in FIG. 19E has a high and broad proximal section with proximal flanges 502 and a round distal end. The adhesive patch 503 shown in FIG. 19F has a wide shape that is short proximally and has scallops 504 on the sides. The adhesive patch 505 has a second aperture 506 above the primary aperture 507, the second aperture being oval and extending laterally wider than the primary aperture 507, as shown in FIG. 19G. The adhesive patch 508 shown in FIG. 19H has a middle set of flanges 509 on each side and a lower set of flanges 510 near the distal end of the patch 508. FIG. 19I illustrates yet another embodiment wherein the adhesive patch 512 has a large, bulbous proximal section 511 and a pair of lateral tabs 513 next to the aperture 514. Similarly, the adhesive patch 515 shown in FIG. 19J has a bulbous proximal section 516 and large tabs 517 extending laterally. The adhesive patch 518 shown in FIG. 19K has a butterfly shape having large proximal wings 519, smaller pointed distal wings 520, and a keyhole-shaped aperture 521. Finally, FIG. 19L shows an adhesive patch 522 which has a proximal slit 523 along a top edge and a distal slit 526 along a bottom edge, an aperture 525 having an oval shape, and a narrow central section 524 on both sides of the aperture 525.

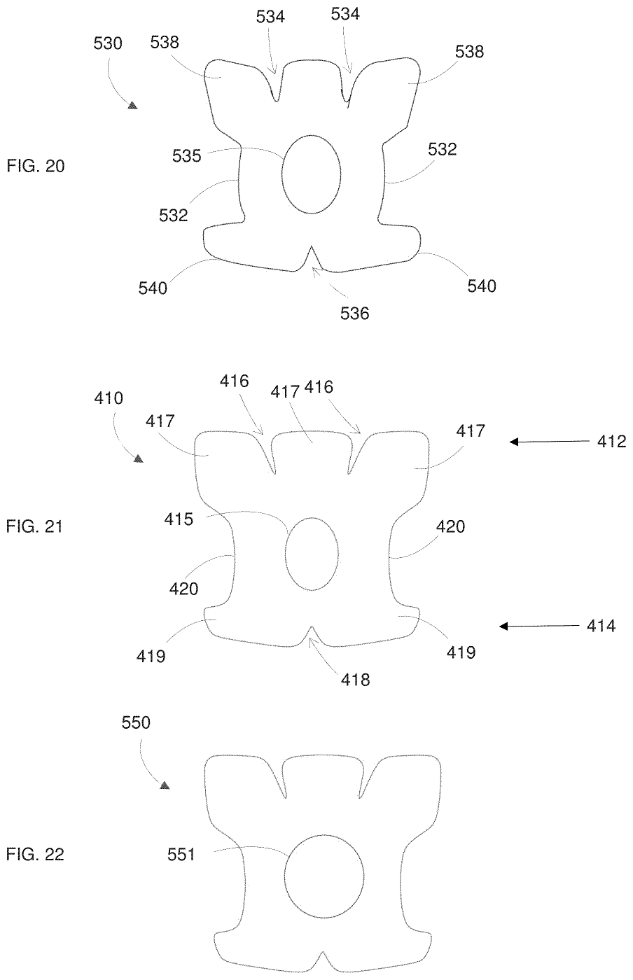

Now with reference to FIG. 20 which shows the adhesive patch of FIGS. 15A-B described above, along with two variations thereof. The adhesive patch 530 of FIG. 20 has an oval aperture 535, a proximal end having two proximal slits 534, a distal end having a slit 536, and a central section 532 on either side of the aperture 535. The central section 532 has a convex base and together with the proximal slits 534, delineates proximal wings 538. The distal edge has a slit 536, and together with the central section 532 delineates distal wings 540.

FIG. 21 shows the same adhesive patch 410 as shown in FIG. 15A-B, but in a planar view to more accurately display the shape and proportions. The adhesive patch 410 has an oval aperture 415, a wide proximal end 412, and a wide distal end 414. The adhesive patch 410 has a narrowed central section 424 defined by concave sidewalls 420. The proximal end 412 may have one or more slits 416 dividing the adhesive patch 410 into proximal tabs 417. Likewise, the distal end 414 may have a slit 418 dividing the adhesive patch 410 into distal tabs 419. The proximal end 412 and the distal end 414 attach to the suprapubic region and scrotum respectively while the central section 413 lays around the penile shaft.

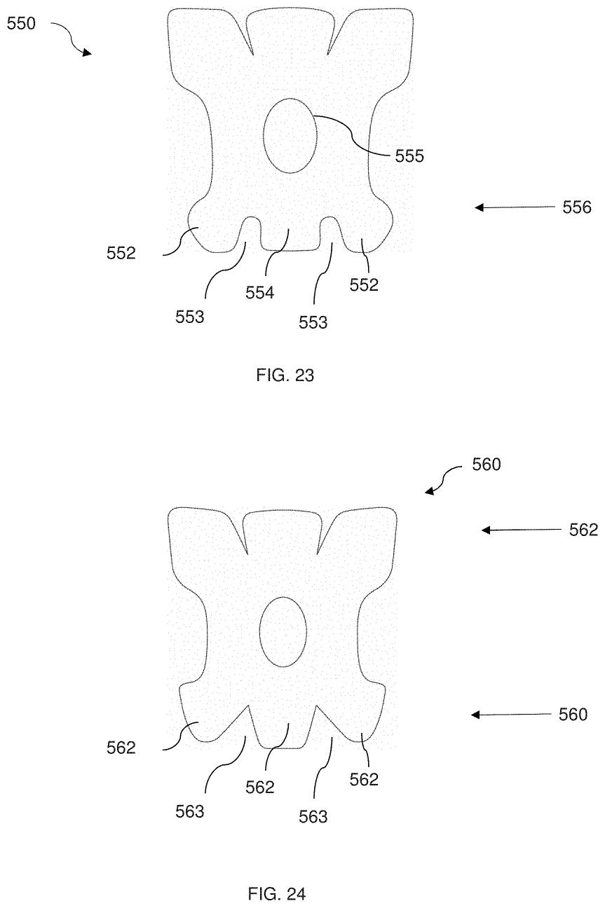

Another embodiment of an adhesive patch 550 is shown in FIG. 22; the patch 550 is otherwise the same as the previously described embodiment shown in FIG. 21, except that it has an aperture 551 which is circular. FIG. 23 illustrates another embodiment of an adhesive patch 550 having an oval aperture 555, wherein the distal end 556 is provided with a plurality of slits 553, dividing the distal end 556 into distal tabs 552, 554. The additional number of distal tabs promotes secure fastening of the adhesive patch 550. FIG. 34 illustrates another embodiment of an adhesive patch 560 having an oval aperture 561B, wherein distal end 559 is provided with a plurality of slits 563 dividing said distal end 560 into distal tabs 562. The shape of the distal tabs 562 illustrated in FIG. 34 are a variation of the shapes of the distal tabs of FIG. 33, having a more open “V” shape, for improving adhesion to (or fastening over) the suprapubic region.

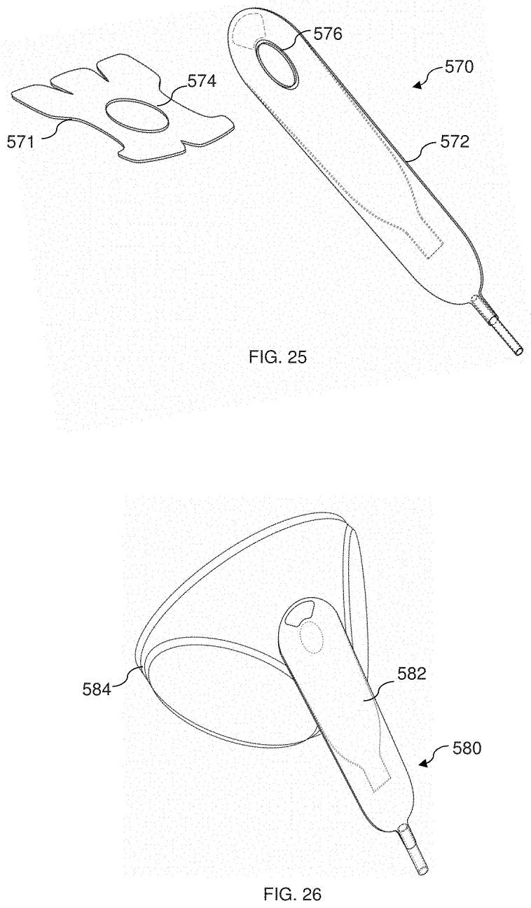

In some embodiments (for example, the urine removal device 570 of FIG. 25), an adhesive patch 571 may be removably attached to the pouch 572. That is, the adhesive patch 571 may be connected to the pouch 572 via an interface that may use threads, a twist-lock (¼ or ½ turn for example), an interfacing male/female flange pair 574 and 576, or any suitable interfacing component pair (comprising first and second interfacing components configured that interface with each other). The pouch 572 may have a corresponding interfacing member or receiving member 576 such as a flange to match a connector 574 on the adhesive patch 571. A detachable interface of this kind allows the operator to remove the pouch for cleaning or replacement while leaving the patch 571 secured via adhesive around the penis, which may save the operator time and effort while preventing any discomfort to the patient that may be incurred while removing and applying the adhesive patch.

FIG. 26 illustrates an embodiment of the urine removal device 580 that is capable of being affixed to clothing (for example, a diaper or undergarment 584 worn by a patient). As would be apparent from FIG. 26 that this may in an embodiment be achieved by incorporating the interfacing mechanism that has been discussed in connection with FIG. 25. In the embodiment illustrated in FIG. 36 the undergarment 584 to which the urine removal device 580 is intended to be affixed is provided with one of an interfacing component pair, or any other suitable interface, while the pouch 582 may have the other of the interfacing component pair (comprising first and second interfacing components configured to interface with each other) which enables the urine removal device 580 and the undergarment 584 to be removably interfaced. While the interfacing components are hidden in FIG. 26, they are similar to that shown in FIG. 25, namely, the first interfacing component may be affixed to the article of clothing, while the second interfacing component may be affixed to the pouch 582. By providing an aperture within the interfacing component (that is affixed to the article of clothing), and by providing affixing the second interfacing component onto the pouch 582, in a manner that it surrounds the aperture formed within pouch 582, the patient's penis can pass through such aperture and the interfacing component pair and into pouch 582 through the aperture provided on pouch 582. The interfacing component pair of FIGS. 25 and 26 may be configured to provide a fluid tight interface between the components.

### Method of Use

In use, a user may align the head of a penis with the aperture within the device 1, such that positioning of the device 1 over the penis will introduce the penis at least partially into the first fluid compartment of the pouch 2. The device may subsequently be moved proximally toward the user such that adhesive patch is in contact with or substantially near the root of the penis and surrounding perineal skin. A release liner may be removed from the adhesive layer to expose the inner surface (not shown), which is sticky or tacky, and press the adhesive patch against the skin adheres the inner surface of the adhesive layer such that it resides flush with the user. If required, proximal tabs and distal tabs may be maneuvered either during adhesion or afterward to assist in customizing the adhesive fit with the user. Next, a drain tube may be temporarily and removably coupled with an outlet conduit member 24.

The devices described in embodiments herein provide for urine removal devices that may be used by patients or other users in the prone position, lying sideways, or sitting because the designs are effective at containing urine without leaking and evacuating urine quickly away from the anatomy.

While the invention disclosed herein has been particularly shown and described with references to example embodiments thereof, it will be understood by those skilled in the art that various changes in form and details may be made therein without departing from the scope of the invention encompassed by the appended claims. Furthermore, while several embodiments are described, the scope of the embodiments should not be construed to be limited to those set forth herein. While the above is a description of certain embodiments, various alternatives, modifications, and equivalents may be used. The various features of the embodiments disclosed herein may be combined or substituted with one another. That is, each of the components of the various embodiments may be combined with each other and that the components of one embodiment may be used with the components of another embodiment. Therefore, the above description should not be taken as limiting the scope of the invention which is defined by the appended claims.

What is claimed is:

1. A urine removal device comprising:
a flexible pouch having a proximal end and a distal end;
an aperture disposed on an external surface of the pouch, wherein the aperture is capable of receiving at least a part of a penis; and
an outlet conduit member affixed to the flexible pouch, the outlet conduit member comprising a fluid inlet, a fluid outlet, and a lumen connecting the fluid inlet to the fluid outlet;
wherein:
the flexible pouch comprises a fluid tight first fluid compartment, a fluid tight second fluid compartment and a first fluid transmission passageway interconnecting the first fluid compartment and the second fluid compartment;
the aperture disposed on the external surface of the pouch is an aperture into the first fluid compartment;
the first fluid transmission passageway comprises a first thin-film unidirectional fluid flow valve that permits fluid flow from the first fluid compartment to the second fluid compartment and that interferes with fluid reflux from the second fluid compartment to the first fluid compartment; and
the first fluid compartment is positioned closer to the proximal end of the pouch and further from the distal end of the pouch; and
the second fluid compartment is positioned closer to the distal end of the pouch and further from the proximal end of the pouch.

2. The urine removal device as claimed in claim 1, wherein the thin-film unidirectional fluid flow valve is a flutter valve.

3. The urine removal device as claimed in claim 1, wherein the fluid inlet of the outlet conduit member is located within the second fluid compartment and the fluid outlet of the outlet conduit member is configured for coupling with a fluid collection receptacle.

4. The urine removal device as claimed in claim 1, wherein:
the first fluid transmission passageway is sized to enable fluid flow from the first fluid compartment to the second fluid compartment at a first flow rate;
the outlet conduit member is sized to enable fluid flow out from the second fluid compartment at a second flow rate; and
the first flow rate is greater than the second flow rate.

5. The urine removal device as claimed in claim 1, wherein the first fluid transmission passageway is sized to enable fluid flow from the first fluid compartment to the second fluid compartment at a flow rate of 10 ml per min or more.

6. The urine removal device as claimed in claim 1, wherein the first fluid transmission passageway comprises a fluid passageway inlet and a fluid passageway outlet, each positioned between the aperture and the fluid inlet of the outlet conduit member.

7. The urine removal device as claimed in claim 1, wherein external peripheral boundaries of the first fluid compartment are located partially or wholly within external peripheral boundaries of the second fluid compartment.

8. The urine removal device as claimed in claim 1, comprising an air exchanger that is gas permeable and liquid impermeable, and that is configured to:
permit air exchange from outside the pouch and into the first fluid compartment, and from the first fluid compartment to outside the pouch.

9. The urine removal device as claimed in claim 1, comprising a fold-resistant feature on the outlet conduit member, wherein the fold-resistant feature is configured to resist the collapse of a lumen defined by the outlet conduit member.

10. The urine removal device as claimed in claim 9, wherein the fold-resistant feature comprises one or more accordion pleats or bellows folds.

11. The urine removal device as claimed in claim 1, wherein a plurality of flow directors are disposed between at least two internal surfaces of the pouch or the first fluid compartment or the second fluid compartment, such that when the two internal surfaces are adjacent each other, channels are created therebetween.

12. The urine removal device as claimed in claim 1, wherein the fluid inlet of the outlet conduit member is positioned between a fluid outlet of the first fluid transmission passageway and the distal end of the pouch.

13. The urine removal device as claimed in claim 1, wherein the outlet conduit member is coupled to the fluid collection receptacle by a drain tube, wherein the drain tube has one or more capillary structures therewithin.

14. The urine removal device as claimed in claim 13, wherein an open-cell foam assembly is positioned as a fluid transmission intermediate between the outlet conduit member and the drain tube, wherein the open-cell foam assembly defines a plurality of fluid channels, each of said fluid channels interconnecting the outlet conduit member and the drain tube.

15. The urine removal device as claimed in claim 14, wherein a suction bulb is positioned as a fluid transmission intermediate between the outlet conduit member and the drain tube, and wherein the suction bulb:
has a fluid inlet coupled with the outlet conduit member; and
a fluid outlet coupled with an inlet of the fluid collection receptacle.

16. The urine removal device as claimed in claim 1, comprising a fluid tight third fluid compartment wherein:
the third fluid compartment is positioned as an intermediate fluid compartment between the second fluid compartment and the outlet conduit member;
a second fluid transmission passageway interconnects the second fluid compartment and the third fluid compartment, the second fluid transmission passageway comprises a second thin-film unidirectional fluid flow valve that permits fluid flow from the second fluid compartment to the third fluid compartment and that interferes with fluid reflux from the third fluid compartment to the second fluid compartment; and
the fluid inlet of the outlet conduit member is located within the third fluid compartment and the fluid outlet of the outlet conduit member is configured for coupling with a fluid collection receptacle.

17. The urine removal device as claimed in claim 16, wherein the second thin-film unidirectional fluid flow valve is a flutter valve.

18. The urine removal device as claimed in claim 1, comprising an adhesive patch attached to the flexible pouch, the adhesive patch configured for fastening the flexible pouch to a patient's suprapubic region.

19. A urine removal device comprising:
a flexible pouch having a proximal end and a distal end;
an aperture disposed on an external surface of the pouch, wherein the aperture is capable of receiving at least a part of a penis; and
an outlet conduit member affixed to the flexible pouch, the outlet conduit member comprising a fluid inlet, a fluid outlet, and a lumen connecting the fluid inlet to the fluid outlet;
wherein:
the flexible pouch comprises:
a fluid tight first fluid compartment;
a fluid tight second fluid compartment;
a first fluid transmission passageway interconnecting the first fluid compartment and the second fluid compartment;
a fluid tight third fluid compartment positioned as an intermediate fluid compartment between the second fluid compartment and the outlet conduit member;
a second fluid transmission passageway interconnecting the second fluid compartment and the third fluid compartment;
wherein:
the aperture disposed on the external surface of the pouch is an aperture into the first fluid compartment;
the first fluid transmission passageway comprises a first thin-film unidirectional fluid flow valve that permits fluid flow from the first fluid compartment to the second fluid compartment and that interferes with fluid reflux from the second fluid compartment to the first fluid compartment;
the second fluid transmission passageway comprises a second thin-film unidirectional fluid flow valve that permits fluid flow from the second fluid compartment to the third fluid compartment and that interferes with fluid reflux from the third fluid compartment to the second fluid compartment;
the first fluid compartment is positioned closer to the proximal end of the pouch and further from the distal end of the pouch;
the second fluid compartment is positioned closer to the distal end of the pouch and further from the proximal end of the pouch; and
the fluid inlet of the outlet conduit member is located within the third fluid compartment and the fluid outlet of the outlet conduit member is configured for coupling with a fluid collection receptacle.
